(19) United States
(12) Patent Application Publication
(10) Pub. No.: US 2026/0263568 A1
(43) Pub. Date: Sep. 10, 2026

(54) AAV-Mediated Gene Therapy

(71) Applicant: The Regents of the University of California, Oakland, CA (US)

(72) Inventor: Ricardo A. Maselli, Davis, CA (US)

(21) Appl. No.: 19/167,310

(22) PCT Filed: Mar. 20, 2024
(86) PCT No.: PCT/US2024/020783

§ 371 Date: Sep. 19, 2025

Related U.S. Application Data
(60) Provisional application No. 63/491,214, filed on Mar. 20, 2023.

(51) Int. Cl. A61K 38/45; A61K 48/00; A61P 21/04; C12N 15/86

(52) U.S. Cl. CPC A61K 38/45; A61K 48/0041; A61K 48/0058; A61K 48/0075; A61P 21/04; C12N 15/86; C12N 2750/14143

(57) ABSTRACT

The present disclosure provides methods and compositions for treating a subject in need thereof by providing a gene encoding choline acetyltransferase (ChAT) to the subject via recombinant AAV virions. The subject may have or may develop a neuromuscular junction (NMJ) disorder (i.e., a myasthenic disorder) such as a presynaptic congenital myasthenic syndrome (CMS) caused by mutations in the CHAT gene. The subject may be identified on the basis of a genetic test, an electrodiagnostic test (e.g., electromyography [EMG]), a blood test (e.g., an antibody serology test), and/or NMJ disorder symptoms, e.g., episodic apneas. The methods and compositions are also useful in increasing ChAT activity in a subject having a deficiency of ChAT activity in their central nervous system (CNS).

### CROSS-REFERENCE

Pursuant to 35 U.S.C. § 119 (e), this application claims priority to the filing date of U.S. Provisional Patent Application Ser. No. 63/491,214, filed on Mar. 20, 2023, the disclosure of which application is herein incorporated by reference in its entirety.

### REFERENCE TO AN ELECTRONIC SEQUENCE LISTING

The contents of the electronic sequence listing (UCDV-373WO_SEQ_LIST_FINAL.xml; Size: 22,440 bytes; and Date of Creation: Mar. 20, 2024) are herein incorporated by reference in their entirety.

### INTRODUCTION

Congenital myasthenic syndromes (CMS) are a heterogeneous group of diseases caused by single-gene defects that are often characterized by muscle weakness and fatigability [1] (Hum Mutat. 2015 September; 36 (9): 881-93). There is an extraordinary variability in the degree of severity of these diseases. For example, symptoms of CMS can range from minimal weakness such as, e.g., weakness only becoming apparent after a prolonged recovery from anesthesia to severe weakness requiring tracheostomy, continuous ventilatory support and tube feeding. While medications such as pyridostigmine, amifampridine and albuterol can ameliorate symptoms in the moderate forms, they are of limited help in the serious variants of CMS. Severe motor deficit is particularly common in presynaptic forms of CMS [2] (Neuron. 2002 Nov. 14; 36 (4): 635-48; Cell Cycle. 2009 Dec. 15; 8 (24): 4017-8). Congenital deficiency of choline acetyltransferase (ChAT) linked to the CHAT gene is the most common presynaptic CMS [1-5] (Hum Gene Ther. 2011 September; 22 (9): 1143-53). The association of this syndrome with dreadful episodes of apnea and sudden infant death syndrome was established as early as 1975 [6] (Hum Gene Ther. 2013 May; 24 (5): 526-32). Further, it has more recently been recognized that CMS due to CHAT mutations can result in extreme degrees of muscle weakness and the requirement of continuous ventilatory support [7,8]. Thus, there is a need for methods for treating serious variants of CMS, such as presynaptic CMS caused by CHAT mutations. The present disclosure fulfills these and other needs.

### SUMMARY

The present disclosure provides methods and compositions for treating a subject in need thereof by providing a gene encoding choline acetyltransferase (ChAT) to the subject via recombinant AAV virions. The subject may have or may develop a neuromuscular junction (NMJ) disorder (i.e., a myasthenic disorder) such as a presynaptic congenital myasthenic syndrome (CMS) due to ChAT deficiency at the neuromuscular junction. The subject may be identified on the basis of a genetic test, an electrodiagnostic test (e.g., electromyography [EMG]), a blood test (e.g., an antibody serology test), and/or NMJ disorder symptoms, e.g., episodic apneas. The methods and compositions are also useful in increasing ChAT activity in a subject having a deficiency of ChAT activity in their central nervous system (CNS).

In certain aspects, the subject may have a deficiency of acetylcholine (ACh) due to ChAT deficiency in their central nervous system (CNS). The subject may have a condition other than a presynaptic CMS linked or associated with the CHAT gene, e.g., other types of presynaptic CMS, myasthenia gravis (MG), and/or Alzheimer's disease (AD) or other types of dementia and the transfer of the ChAT encoding gene into the subject results in the prevention and/or reduction in severity of symptoms manifested in the subject.

### BRIEF DESCRIPTION OF THE FIGURES

FIG. 1 provides the structure of human ChAT and identified mutations. The catalytic histidine is depicted in red, and the active-site tunnel is denoted with an asterisk. The structure is represented in a ribbon diagram with α-helices in blue, β-sheet in green, coils in orange, and mutated residues depicted in magenta.

FIG. 2 provides a diagram of the mouse Chat gene depicting the position of the LoxP sites used to generate the Chat knockout (KO) mouse model.

FIGS. 3A to 3B provide the breeding strategy and demonstrate the genotype of mice used to produce the conditional ChAT KO mouse model. (A) a breeding strategy used to produce ChAT^{flox/flox}/Cre-ER^{T2/WT} mice. (B) gel electrophoresis bands demonstrating the genotype of various mice progeny.

FIG. 4 provides an experimental design performed on WT mice, WT+Tx mice, MT+Tx mice, and MT+AAV+Tx.

FIGS. 5A to 5G provide the results of a survival curve and motor performances of MT+AAV+Tx mice, MT+Tx mice, and WT mice. (A) the Kaplan-Meier analysis and log-rank tests used to analyze mice survival. (B) weight measurements during the testing period. (C) (D) the results of a rotarod performance test. (E) grip strength averages taken during the testing period of four weeks. (F) EMG results comparing compound muscle action potential (CMAP) amplitudes before and after a long run of nerve stimulation at 30 Hz for 30 seconds. (G) representative examples of EMG recordings.

FIGS. 6A to 6H provide immunohistochemistry results of ChAT expression in motor neurons of the lumbar spinal cord of MT+AAV+Tx mice, MT+Tx mice, and WT mice. (A) expression of Chat in motor neurons of a WT mouse. (B) Expression of Chat in motor neurons of a MT+AAV+Tx mouse. (C) expression of Chat in motor neurons of a MT+Tx mouse. (D) a bar graph showing the average emission of Alexa Fluor 594 in motor neurons of MT+AAV+Tx mice, MT+Tx mice, and WT mice. (E-G) examples of ChAT IF in the cervical spines of a WT (E), an AAV-treated (F), and a nontreated (G) mice. (H) a bar graph showing the expression of ChAT in cervical spines of AAV-Tx mice in comparison with the Tx and WT.

FIG. 7 depicts pathologic assessment of long surviving mice after neonatal injection of AAV9-CHAT.

FIG. 8 illustrates mortality of mutant mice injected and noninjected with AAV9-CHAT at postnatal (P) day 28 after the induction of disease by injection of tamoxifen at P11.

FIG. 9 provides a scatterplot showing great variability and widespread distribution of the viral genome inside and outside the CNS in Chat^{flox/flox}-Cre-ER^{T2} mice injected with AAV9-CHAT.

FIG. 10 provides a column scatter graph illustrating gene expression in the brain, spinal cord, and liver of AAV-treated and untreated mice.

### DETAILED DESCRIPTION

The present disclosure provides methods and compositions for treating a subject in need thereof by providing a gene encoding choline acetyltransferase (ChAT) to the subject via recombinant AAV virions. The subject may have or may develop a neuromuscular junction (NMJ) disorder (i.e., a myasthenic disorder) such as a presynaptic congenital myasthenic syndrome (CMS) associated or linked with the CHAT gene. The subject may be identified on the basis of a genetic test, an electrodiagnostic test (e.g., electromyography [EMG]), a blood test (e.g., an antibody serology test), and/or NMJ disorder symptoms, e.g., episodic apneas. The methods and compositions are also useful in increasing ChAT activity in a subject having a deficiency of ChAT activity in their central nervous system (CNS) in order to treat other various CNS related diseases and conditions.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. Thus, for example, reference to “a rAAV virion” includes a plurality of such rAAV virions and reference to “the vector” includes reference to one or more vectors, “a mutation” refers to one or more mutations, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. To the extent such publications may set out definitions of a term that conflicts with the explicit or implicit definition of the present disclosure, the definition of the present disclosure controls.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Definitions

“AAV” is an abbreviation for adeno-associated virus and may be used to refer to the virus itself or derivatives thereof, such as, engineered AAVs. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes all AAV serotypes as well as AAV vectors based on the combination of different serotypes (also referred to as “hybrid AAV vectors” or “pseudotype AAV vectors”). In one aspect, the AAV vector is an AAV Vector with a serotype selected from AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10) (AAV-Rh10), AAV type 11 (AAV-11), avian AAV, bovine AAV, canine AAV, equine AAV, primate AAV, non-primate AAV, ovine AAV, AAV-7m8, AAV-6.2, AAV-Dj, AAV-DJ/8, AAV2-retro, AAV2-QuadYF and AAV2.7m8, and combinations thereof. See, e.g., Mori et al. (2004) Virology 330:375. The synthetic AAV variant AAV-7m8 is described, for example, in Dalkara et al. See Transl Med 2013, 5 (189): 189ra76. The term “AAV” also includes chimeric AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc. AAV also encompasses mixed serotype capsids, AAV-PHP.B, AAV-PHP.B2, AAV-PHP.B3, AAV-PHP.A, AAV-PHP.eB, AAV-PHP.eS, evolved capsids that are less immunogenic to mice and humans, and variants thereof. The term “AAV” also includes both anterograde and retrograde embodiments of any of the AAVs mentioned above.

An “rAAV vector” as used herein refers to an AAV vector including a polynucleotide sequence not of AAV origin (i.e., a polynucleotide heterologous to AAV), typically a sequence of interest for delivery to a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins) and an encapsidated polynucleotide rAAV vector. If the particle includes a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). “Helper virus function” may be provided in a number of ways, including by providing helper virus or providing, for example, polynucleotide sequences encoding the requisite function(s) to a producer cell in trans.

An “infectious” virus or viral particle is one that includes a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome (vg) copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus includes a nucleotide sequence encoding GFP), where GFP is produced in a cell infected with the viral particle and is detected and/or measured; or the measurement of a produced protein, for example by an enzyme-linked immunosorbent assay (ELISA). Viral infectivity can be expressed as the ratio of infectious viral particles to total viral particles. Methods of determining the ratio of infectious viral particle to total viral particle are known in the art.

A “replication-competent” virus (e.g. a replication-competent AAV) refers to a phenotypically wild-type virus that is infectious and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 102 rAAV particles, less than about 1 rcAAV per 10⁴ rAAV particles, less than about 1 rcAAV per 108 rAAV particles, less than about 1 rcAAV per 10¹² rAAV particles, or no rcAAV).

The term “polynucleotide” or “nucleic acid” refers to a polymeric form of nucleotides of any length, including deoxyribonucleotides or ribonucleotides, or analogs thereof. A polynucleotide may include modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses the single-stranded form, the double-stranded form, and each of two complementary single-stranded forms known or predicted to make up the double-stranded form.

The terms “polypeptide” and “protein” are used interchangeably herein and refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues, etc.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST or FASTA. Of particular interest are alignment programs that permit gaps in the sequence. Of interest is the BestFit program using the local homology algorithm of Smith Waterman (Advances in Applied Mathematics 2:482-489 (1981) to determine sequence identity.

A “gene” refers to a polynucleotide containing at least one open reading frame that is capable of encoding a particular protein after being transcribed and translated.

“Conservative amino acid substitution” refers to a substitution of one amino acid residue for another sharing chemical and physical properties of the amino acid side chain (e.g., charge, size, hydrophobicity/hydrophilicity). “Conservative substitutions” are intended to include substitution within the following groups of amino acid residues: gly, ala; val, ile, leu; asp, glu; asn, gln; ser, thr; lys, arg; and phe, tyr.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct. A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also includes control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally occurring, wild-type AAV, and the encoded heterologous gene product is a gene product not normally encoded by a naturally-occurring, wild-type AAV.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

“Treatment,” as used herein, covers any treatment of a disease in a mammal, particularly in a human, and includes: a) inhibiting the disease, i.e., arresting or slowing its development; and (b) relieving the disease, i.e., causing regression of the disease or its clinical symptoms. One form of treatment includes prevention of occurrence of a disease or delay in onset of the disease and/or reduced severity of the disease after onset of the disease in a subject which may be predisposed to the disease or at risk of acquiring the disease but has not yet been diagnosed as having it.

The terms “individual,” “host,” “subject,” and “patient” are used interchangeably herein, and refer to a mammal, including, but not limited to, human and non-human primates, including simians and humans; mammalian sport animals (e.g., horses, camels, etc.); mammalian farm animals (e.g., sheep, goats, cows, etc.); mammalian pets (dogs, cats, etc.); and rodents (e.g., mice, rats, etc.). In some cases, the individual is a human.

The term “pharmaceutically acceptable” refers to a non-toxic material which preferably does not interfere with the action of the active ingredient of the pharmaceutical composition. In particular, the term “pharmaceutically acceptable” means that the subject substance has been approved by a governmental regulatory agency for use in animals, and particularly humans, or in U.S. Pat. Pharmacopoeia, European Pharmacopoeia or other recognized pharmacopoeias for use in animals and in particular humans.

### Methods

The present disclosure provides a method for treating a neuromuscular junction (NMJ) disorder (i.e., a myasthenic disorder) such as a presynaptic congenital myasthenic syndrome (CMS) associated or linked with the CHAT gene in a subject. The method involves transfer of a nucleic acid encoding choline acetyltransferase (ChAT) via recombinant AAV virions into a subject in need thereof. The present disclosure also encompasses identification of the subject in need of receiving the nucleic acid. Various steps and aspects of the method will now be described in greater detail below.

The CHAT gene encodes the enzyme choline acetyltransferase (ChAT), which catalyzes the transfer of an acetyl group from acetyl-CoA to choline for the synthesis of acetylcholine (ACh), a neurochemical that has a wide variety of functions in the brain and other organ systems of the body. For example, the role of ACh as a neurotransmitter that regulates signal transduction at the neuromuscular junction is of specific interest in this disclosure. Patients having an NMJ disorder (i.e., a myasthenic disorder) may experience symptoms such as muscle weakness and fatigue. In cases where the NMJ disorder results in a deficiency of ACh or a reduced ability of the central nervous system (CNS) to synthesize ACh, such as, e.g., CMS associated or linked with the CHAT gene, the subject may exhibit symptoms including, but not limited to, ptosis, hypotonia, muscle fatigue, loss of postural tone, respiratory apneas (e.g., episodic apneas), temperature-dependent weakness (such as, e.g., “swimming pool weakness”), facial and eye muscle weakness, soft palate weakness requiring gastric tube, severe generalized weakness, respiratory failure requiring tracheotomy and mechanical ventilation, and/or mental developmental delays with a wide range of severity. By associated or linked with is meant the disease or condition is correlated with or has a relationship to the gene of interest (i.e., CHAT). For example, the disease or condition may be caused by mutations to the gene of interest (or, e.g., a control element thereof), symptoms of the disease or condition may be exacerbated by mutations to the gene, and/or a diagnosis of the disease or condition (including, e.g., the severity thereof) in a subject may be made, at least in part, by determining the genotype of the gene.

In some cases, the disease may be presynaptic CMS associated or linked with the CHAT gene. In these cases, mutations of the CHAT gene may cause the CMS and a diagnosis of the CMS (e.g., including the severity of the CMS) may be made, at least in part, by determining the genotype or specific mutation(s) of the subject's endogenous CHAT gene. Patients having CMS associated or linked with the CHAT gene (CHAT-CMS) may experience any of the above symptoms, including episodic apneas, and may have a reduced life span with some patients dying in utero, infancy, or early childhood. CHAT-CMS patients may survive to adulthood but may be wheelchair bound, requiring a gastric tube and mechanical ventilatory support for life. In other cases, the subject may have a disease or condition linked or associated with a deficiency of acetylcholine (ACh) or a reduced ability to synthesize ACh in their central nervous system (CNS) apart from, or in addition to, CHAT-CMS. For example, the subject may have other types of presynaptic CMS, myasthenia gravis (MG), and/or Alzheimer's disease (AD) or other types of dementia and the transfer of the ChAT encoding gene into the subject results in the prevention and/or reduction in severity of symptoms manifested in the subject from these diseases.

A subject who may be treated with the methods disclosed herein may be a newborn, an infant, a toddler, a child, a teenager, or an adult. In some cases, the subject may be eight years of age (i.e., from their birth) or younger, such as three years of age or younger, or one year or younger, or one month or younger, or one day or younger. In some instances, the subject may be a fetus in utero and may be forty weeks of age (i.e., from conception) or younger, such as twenty-six weeks of age or younger, or thirteen weeks of age or younger, or eight weeks of age or younger. The subject may exhibit one or more symptoms of an NMJ disorder and/or have a mutation associated or linked with the NMJ disorder. In some aspects, a subject receiving a rAAV virion according to the present disclosure may be treated for an NMJ disorder, based on, e.g., a genetic test and/or a blood test. The genetic test may determine the genotype or specific mutation(s) of a gene linked or associated with the NMJ disorder. The blood test may be an antibody serology test such as, e.g., a serology test to determine the presence or level of antibodies targeting chemical structures or body components producing or receiving ACh (e.g., antibodies targeting nicotinic acetylcholine receptors). In some embodiments, the blood test may be an antibody serology test that determines the presence or level of antibodies targeting AAV serotypes included in the rAAV virion used for administering the nucleic acid encoding ChAT. In some instances, a subject receiving a rAAV virion according to the present disclosure may be treated for an NMJ disorder, based on, e.g., a NMJ disorder symptom, a genetic test, and/or an electrodiagnostic test. For example, the subject may receive the rAAV virion based on whether or not they exhibit any of the symptoms described above, or based on the results of an electromyography (EMG) test.

As discussed above, a subject receiving a rAAV virion according to the present disclosure may be treated for an NMJ disorder based on one or more of a genetic test, a blood test (e.g., an antibody serology test), NMJ disorder symptoms, and/or an electrodiagnostic test (e.g., electromyography [EMG]). In some embodiments, the tests and exhibited symptoms are used to determine if the subject has a treatment or condition effectively treated by the rAAV virion of the present disclosure. In some embodiments, the tests are used to determine if the subject may be prone to experiencing side effects or complications as a result of being administered the rAAV virion of the present disclosure.

In some cases, a subject receiving a rAAV virion according to the present disclosure may be treated for CHAT-CMS based on one or more of a genetic test, a blood test (e.g., an antibody serology test or a test to determined endogenous ChAT activity), CHAT-CMS symptoms, and/or an electrodiagnostic test (e.g., electromyography [EMG]). In some cases, the CHAT-CMS symptom exhibited by the patient is respiratory apneas such as episodic apneas. In some cases, the subject may not exhibit symptoms of CHAT-CMS. In some instances, the genetic test determines the genotype or specific mutation(s) of the subject's endogenous CHAT gene (or, e.g., a control element thereof). The mutation(s) include any mutations that may affect the level of ChAT enzymatic activity and/or expression in the subject (e.g., the CNS of the subject). For example, a genetic test may be performed to determine the presence of mutation(s) affecting the expression level of ChAT (i.e., the amount of ChAT enzyme), one or more kinetic properties of ChAT, or the stability of ChAT in the CNS of the subject. In some cases, the subject may be identified as in need of treatment based on the determined presence of one or more mutations selected from the group of Val136Met, Arg186Trp, Val194Leu, Arg207His, Pro211Ala, Arg566Cys, and/or Ser694Cys, wherein the amino acid numbering is based on the amino acid sequence set forth in SEQ ID NO:1. In some cases, the mutation is Val136Met and/or Arg207His. As would be understood from the present disclosure, the treatment described herein is not limited to subjects having a mutation in the CHAT gene, e.g., complete or partial gene deletion and mutations causing alternate splicing but is also applicable to subjects with a ChAT deficiency or any other condition affecting the synthesis of acetylcholine.

The rAAV virion may be administered in a therapeutically effective amount. A “therapeutically effective amount” will fall in a relatively broad range that can be determined through experimentation and/or clinical trials. For example, a therapeutically effective dose can be on the order of from about 10⁶ to about 10¹⁸ of the rAAV virions, e.g., from about 10¹⁰ to 10¹⁵ rAAV virions, from about 10⁶ viral genomes (vg) to about 10¹⁸ vg of the rAAV virions, e.g., from about 10¹⁰ vg to 10¹⁵ vg. In some instances, the weight of the therapeutically effective dose is determined, at least in part, using the weight of the subject. For example, a therapeutically effective dose can be on the order of from about 10⁵ to about 10¹⁷ of the rAAV virions per kilogram the subject weighs, e.g., from about 10⁹ to 10¹⁴ rAAV virions per kg, from about 10⁵ viral genomes (vg) to about 10¹⁷ vg of the rAAV virions per kg, e.g., from about 10⁵ vg to 10¹⁴ vg per kg. Other effective dosages can be readily established by one of ordinary skill in the art through routine trials establishing dose response curves.

In some embodiments, more than one administration (e.g., two, three, four or more administrations) may be employed to achieve the desired level of gene expression. In some cases, the more than one administration is administered at various intervals, e.g., daily, weekly, twice monthly, monthly, every 3 months, every 6 months, yearly, etc. In some cases, multiple administrations are administered over a period of time from 1 month to 2 months, from 2 months to 4 months, from 4 months to 8 months, from 8 months to 12 months, from 1 year to 2 years, from 2 years to 5 years, or more than 5 years.

In some embodiments the AAV serotype is selected from: AAV1, AAV2, AAV3, AAV4, AAV5, AAV6, AAV7, AAV8, AAV9, and AAVrh.10. In other embodiments the AAV serotype is a variant of an AAV serotype selected from: AAV1, AAV2, AAV3, AAV4, AAV5, AAV6, AAV7, AAV8, AAV9, and AAVrh. 10. In some instances, the rAAV virion includes AAV serotype 9 capsid proteins.

The nucleic acid encoding ChAT is flanked by AAV inverted terminal repeats (ITRs), e.g., AAV1 ITRs, AAV2 ITRs, AAV3 ITRs, AAV4 ITRs, AAV5 ITRs, AAV6 ITRs, AAV7 ITRs, AAV8 ITRs, AAV9 ITRs, AAV102 ITRs, or AAV11 ITRs. In certain aspects, the nucleic acid encoding ChAT is flanked by AAV2 ITRs, AAV3 ITRs, or AAV9 ITRs.

In certain aspects, the subject may be human and the ChAT may be a human ChAT or, e.g., a non-human ChAT that, when expressed or introduced into the CNS of a human, has a similar level of ChAT activity as a human ChAT. In some embodiments, the ChAT may be a human ChAT. The nucleic acid present in the rAAV may encode a human ChAT comprising an amino acid sequence that is at least 75%, 80%, 85%, 90%, 95%, 96%, 97%, 98%, or 99% or a 100% identical to the amino acid sequence:

(SEQ ID NO: 1)
MAAKTPSSEESGLPKLPVPPLQQTLATYLQCMRHLVSEEQFRKSQAIVQ
QFGAPGGLGETLQQKLLERQEKTANWVSEYWLNDMYLNNRLALPVNSSP
AVIFARQHFPGTDDQLRFAASLISGVLSYKALLDSHSIPTDCAKGQLSG
QPLCMKQYYGLFSSYRLPGHTQDTLVAQNSSIMPEPEHVIVACCNQFFV
LDVVINFRRLSEGDLFTQLRKIVKMASNEDERLPPIGLLTSDGRSEWAE
ARTVLVKDSTNRDSLDMIERCICLVCLDAPGGVELSDTHRALQLLHGGG
YSKNGANRWYDKSLQFVVGRDGTCGVVCEHSPFDGIVLVQCTEHLLKHM
TQSSRKLIRADSVSELPAPRRLRWKCSPEIQGHLASSAEKLQRIVKNLD
FIVYKFDNYGKTFIKKQKCSPDAFIQVALQLAFYRLHRRLVPTYESASI
RRFQEGRVDNIRSATPEALAFVRAVTDHKAAVPASEKLLLLKDAIRAQT
AYTVMAITGMAIDNHLLALRELARAMCKELPEMFMDETYLMSNRFVLST
SQVPTTTEMFCCYGPVVPNGYGACYNPQPETILFCISSFHSCKETSSSK
FAKAVEESLIDMRDLCSLLPPTESKPLATKEKATRPSQGHQP.

As used herein, human ChAT refers to a polypeptide comprising the amino acid sequence set forth above as SEQ ID NO:1, a functional fragment thereof, or a polypeptide having ChAT activity when expressed or introduced into the CNS of a human and comprising an amino acid sequence at least 75% identical (e.g., at least 80%, at least 85%, at least 90%, at least 95%, at least 96%, at least 97%, at least 98%, or at least 99%) to the amino acid sequence set forth in SEQ ID NO:1. In certain aspects, the nucleic acid encoding the human ChAT may be a codon-optimized sequence, where the codons are codons from highly expressed human genes. In some cases, one or more conservative substitutions may occur with little to no effect on ChAT activity (e.g., enzyme expression, kinetics, or stability). In some instances, one or more semi-conservative substitutions or non-conservative substitutions may occur a distance away from the active-site tunnel and active site of the ChAT enzyme.

In certain aspects, the ChAT polypeptide may be engineered to enhance ChAT activity when expressed or introduced into the CNS of a human. For example, human ChAT may be engineered for increased expression, stability, or more favorable kinetics (i.e., leading to an increase in the reaction rate of ChAT facilitated ACh production), through one or more of a variety of known protein engineering techniques including, but not limited to, rational and semi-rational design, directed evolution, molecular dynamics and homology modeling, random mutagenesis, DNA shuffling, cell surface display techniques, peptidomimetics, flow cytometry/cell sorting, etc. The human ChAT may be a soluble human ChAT or may be a membrane bound human ChAT. In some cases, the ChAT enzyme may have little to no sequence homology to human ChAT. In these cases, the ChAT enzyme may be created through de novo enzyme engineering. In some embodiments, the ChAT polypeptide may not be derived from humans (i.e., may not be natural to humans) but, when expressed or introduced into the CNS of a human, has at least a similar level of ChAT activity as wild type human ChAT (i.e., the human ChAT set forth in SEQ ID NO:1).

In some embodiments, the ChAT polypeptide (e.g., the human ChAT set forth in SEQ ID NO: 1) includes a signal peptide at the N-terminal of the polypeptide. Signal peptide (SPs) are short amino acid sequences (e.g., typically 16 to 30 residues in length) present at the N-terminus of a newly synthesized polypeptide that can direct localization of the polypeptide. SPs are typically cleaved off of the end of polypeptides, e.g., by signal peptidase. In some embodiments, the ChAT polypeptide includes a signal peptide for localization of the ChAT polypeptide at the axon terminal of neuronal cells (e.g., cholinergic neurons) in which the polypeptide is expressed. For example, the rAAV may encode a human ChAT comprising a signal peptide at the N-terminal of the ChAT polypeptide that directs the polypeptide to the axon terminal when it is expressed in cholinergic neurons.

Treatment of an NMJ disorder in a subject in need thereof may result in an increase of ChAT activity (i.e., ChAT enzymatic activity) in the CNS of the patient. The increase of ChAT activity may treat a condition in which sufficient level of enzymatically active CHAT is not expressed, e.g., a condition where the CHAT expressed in enzymatically inactive or a condition where the CHAT expressed is enzymatically active but the expression level is below normal or insufficient for normal NMJ function. Treatment of the NMJ disorder in a subject in need thereof may result in an increase of ACh levels in the CNS of the subject, such as the NMJs of the subject. Treatment of the NMJ disorder in a subject in need thereof may result in the prevention and/or reduction in severity of symptoms manifested in the subject. Treatment of the NMJ disorder in a subject in need thereof may result in an increase in the quality of life of the subject. The treatment may increase life span of the subject by at least 1 year or more, e.g., at least 5 years, 10 years, 15 years, or more as compared to subjects with the NMJ disorder not receiving the treatment. In some cases, the treatment may result in the subject having a normal life span or, e.g., a life span unaffected by the NMJ disorder.

The presently disclosed rAAV finds use in increasing ChAT activity in a subject in need thereof. ChAT activity may be measured in a sample from the subject such as a blood sample or a fraction thereof (e.g., serum or plasma), cerebrospinal fluid (CSF), lymphocytes or tissue. The measured level may be compared to a normal level to determine whether a subject has increased ChAT activity. The measured level may be compared to a threshold level that is lower than the normal level to determine whether a subject has increased ChAT activity. The normal and threshold ChAT activity levels may be determined based on ChAT activity measurement in age matched and/or gender matched healthy subjects. The subject may have CHAT-CMS or may have another condition such as other types of presynaptic CMS, myasthenia gravis (MG), and/or Alzheimer's disease (AD) or other types of dementia. The administration of the rAAV may result in an expression level of ChAT activity that is at least 50% of the normal ChAT activity level and may result in a 50% or higher increase in the of ACh levels in the CNS of the subject, such as the NMJs of the subject, as compared to prior to the treatment.

In cases where the subject has CHAT-CMS, treatment of CHAT-CMS in a subject in need thereof may result in an increase of ChAT activity (i.e., ChAT enzymatic activity) in the CNS of the patient, such as the NMJs of the patient. Treatment of CHAT-CMS in a subject in need thereof may result in the prevention and/or reduction in severity of symptoms manifested in the subject such as reductions of apneic episodes or increased time-off mechanical ventilation. Treatment of CHAT-CMS in a subject in need thereof may result in an increase in the quality of life of the subject. For example, treatment may result in the ability to sit independently, ability to overcome gravity with proximal muscles, ability to stand up and ambulate and independency, etc. In some cases, the increase in the quality of life is measured by or determined using a change in the Myasthenia Gravis Activities of Daily Living (MG-ADL) total score or the Quantitative Myasthenia Gravis (QMG) total score. The treatment of CHAT-CMS in the subject may increase life span of the subject by at least 1 year or more, e.g., at least 5 years, 10 years, 15 years, or more as compared to subjects with CHAT-CMS not receiving the treatment. In some cases, the treatment may result in the subject having a normal life span or, e.g., a life span unaffected by CHAT-CMS.

### Recombinant AAVs: Production Methods

Methods for obtaining recombinant AAVs having a desired capsid protein are well known in the art (see, for example, US 2003/0138772, the contents of which are incorporated herein by reference in their entirety). Typically, the methods involve culturing a host cell which contains a nucleic acid sequence encoding an AAV capsid protein or fragment thereof; a functional rep gene; a recombinant AAV vector composed of, AAV inverted terminal repeats (ITRs) and a therapeutic transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 (e.g., E1a and E1b) helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the invention may be delivered to the packaging host cell using any appropriate genetic element (vector). The selected genetic element may be delivered by any suitable method, including those described herein. The methods used to construct any embodiment of this invention are known to those with skill in nucleic acid manipulation and include genetic engineering, recombinant engineering, and synthetic techniques.

In certain embodiments, the recombinant AAVs are produced by triple plasmid transfection method involving transfecting a host cell (e.g., a 293 cell) with (i) an adenovirus helper plasmid (ii) a rep/cap plasmid expressing AAV rep (e.g., rep 2) and AAV cap (e.g., AAV cap9) and (iii) a transgene plasmid, e.g., encoding a therapeutic polypeptide. The adenovirus helper plasmid supports efficient AAV vector production without generating any detectable wild-type AAV virions. The adenovirus helper plasmid encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus. In certain aspects, the host cell may be HEK293T host cell that expresses adenovirus E1a and E1b constitutively. The HEK293T host cell that expresses adenovirus E1a and E1b constitutively may be transfected using the triple transection method involving simultaneous transfection with (i) an adenovirus helper plasmid (ii) a rep/cap plasmid expressing AAV rep (e.g., rep 2) and AAV cap (e.g., AAV cap9) and (iii) a transgene plasmid, e.g., encoding a therapeutic, e.g., ChAT.

In certain embodiments, the AAV rep/cap plasmid may be a pAAV expressing AAV2 rep and AAV9 cap, such as, pAAVrep2cap9 described in Zhu, H. et al., Clin Transl Med. 2023 July; 13 (7): e1326. In certain embodiments, the pAAVrep2cap9 may be used for triple transfection of a HEK293T cell along with a helper plasmid and the transgene encoding plasmid, e.g., as described in Zhu, H. et al.

In some aspects, the invention provides transfected host cells. The term “transfection” is used to refer to the uptake of foreign DNA by a cell, and a cell has been “transfected” when exogenous DNA has been introduced inside the cell membrane. A number of transfection techniques are generally known in the art. Transfection may be achieved for example by infecting a cell with a rAAV harboring a rAAV vector. A “host cell” refers to any cell that harbors, or is capable of harboring, a substance of interest. Often a host cell is a mammalian cell. A host cell may be used as a recipient of an AAV rep/cap construct, an AAV transgene plasmid, e.g., comprising a promoter operably linked with a ChAT encoding gene, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter.

The foregoing methods for packaging recombinant vectors in desired AAV capsids to produce the rAAVs of the present disclosure are not meant to be limiting and other suitable methods will be apparent to the skilled artisan.

### Recombinant AAV Vectors

Recombinant AAV (rAAV) vectors of the present disclosure are typically composed of, at a minimum, a therapeutic transgene, e.g., encoding a ChAT, and its regulatory sequences, and 5′ and 3′ AAV inverted terminal repeats (ITRs). It is this recombinant AAV vector which is packaged into a capsid protein (i.e., included in the rAAV virion) and delivered to a subject. The nucleic acid sequence encoding ChAT is operatively linked to regulatory components in a manner which permits transgene transcription, translation, and/or expression in a cell.

The nucleic acid encoding ChAT may encode any of the ChAT polypeptides (i.e., ChAT enzymes) as discussed above. In cases where the ChAT is human ChAT, the nucleic acid encoding the human ChAT may comprise the following sequence or a sequence comprising at least 50%, at least 60%, at least 70%, at least 75%, at least 80%, at least 85%, at least 90%, at least 95%, at least 97%, at least 98%, or at least 99% sequence identity to the following nucleic acid sequence:

(SEQ ID NO: 2)
ATGGCAGCAAAAACTCCCAGCAGTGAGGAGTCTGGGCTGCCCAAACTGCCCG
TGCCCCCGCTGCAGCAGACCCTGGCCACGTACCTGCAGTGCATGCGACACTTGGTGT
CTGAGGAGCAGTTCAGGAAGAGCCAGGCCATTGTGCAGCAGTTTGGGGCCCCTGGT
GGCCTCGGCGAGACCCTGCAGCAGAAACTCCTGGAGCGGCAGGAGAAGACAGCCA
ACTGGGTGTCTGAGTACTGGCTGAATGACATGTATCTCAACAACCGCCTGGCCCTGC
CTGTCAACTCCAGCCCTGCCGTGATCTTTGCTCGGCAGCACTTCCCTGGCACCGATG
ACCAGCTGAGGTTTGCAGCCAGCCTCATCTCTGGTGTACTCAGCTACAAGGCCCTGC
TGGACAGCCACTCCATTCCCACTGACTGTGCCAAAGGCCAGCTGTCAGGGCAGCCCC
TTTGCATGAAGCAATACTATGGGCTCTTCTCCTCCTACCGGCTCCCCGGCCATACCC
AGGACACGCTGGTGGCTCAGAACAGCAGCATCATGCCGGAGCCTGAGCACGTCATC
GTAGCCTGCTGCAATCAGTTCTTTGTCTTGGATGTTGTCATTAATTTCCGCCGTCTCA
GTGAGGGGGATCTGTTCACTCAGTTGAGAAAGATAGTCAAAATGGCTTCCAACGAG
GACGAGCGTTTGCCTCCAATTGGCCTGCTGACGTCTGACGGGAGGAGCGAGTGGGC
CGAGGCCAGGACGGTCCTCGTGAAAGACTCCACCAACCGGGACTCGCTGGACATGA
TTGAGCGCTGCATCTGCCTTGTATGCCTGGACGCGCCAGGAGGCGTGGAGCTCAGCG
ACACCCACAGGGCACTCCAGCTCCTTCACGGCGGAGGCTACAGCAAGAACGGGGCC
AATCGCTGGTACGACAAGTCCCTGCAGTTTGTGGTGGGCCGAGACGGCACCTGCGGT
GTGGTGTGCGAACACTCCCCATTCGATGGCATCGTCCTGGTGCAGTGCACTGAGCAT
CTGCTCAAGCACATGACGCAGAGCAGCAGGAAGCTGATCCGAGCAGACTCCGTCAG
CGAGCTCCCCGCCCCCCGGAGGCTGCGGTGGAAATGCTCCCCGGAAATTCAAGGCC
ACTTAGCCTCCTCGGCAGAAAAACTTCAACGAATAGTAAAGAACCTTGACTTCATTG
TCTATAAGTTTGACAACTATGGGAAAACATTCATTAAGAAGCAGAAATGCAGCCCT
GATGCCTTCATCCAGGTGGCCCTCCAGCTGGCCTTCTACAGGCTCCATCGAAGACTG
GTGCCCACCTACGAGAGCGCGTCCATCCGCCGATTCCAGGAGGGACGCGTGGACAA
CATCAGATCGGCCACTCCAGAGGCACTGGCTTTTGTGAGAGCCGTGACTGACCACA
AGGCTGCTGTGCCAGCTTCTGAGAAGCTTCTGCTCCTGAAGGATGCCATCCGTGCCC
AGACTGCATACACAGTCATGGCCATAACAGGGATGGCCATTGACAACCACCTGCTG
GCACTGCGGGAGCTGGCCCGGGCCATGTGCAAGGAGCTGCCCGAGATGTTCATGGA
TGAAACCTACCTGATGAGCAACCGGTTTGTCCTCTCCACTAGCCAGGTGCCCACAAC
CACGGAGATGTTCTGCTGCTATGGTCCTGTGGTCCCAAATGGGTATGGTGCCTGCTA
CAACCCCCAGCCAGAGACCATCCTTTTCTGCATCTCTAGCTTTCACAGCTGCAAAGA
GACTTCTTCTAGCAAGTTTGCAAAAGCTGTGGAAGAAAGCCTCATTGACATGAGAG
ACCTCTGCAGTCTGCTGCCGCCTACTGAGAGCAAGCCATTGGCAACAAAGGAAAAA
GCCACGAGGCCCAGCCAGGGACACCAACCT.

In some cases, the nucleic acid encoding human ChAT may be a codon optimized version of the nucleotide sequence set forth in SEQ ID NO:2. For example, one or more codons in SEQ ID NO: 2 may be replaced with codon(s) from highly expressed human genes to increase expression of the ChAT in a human subject receiving the rAAV. In some cases, the nucleic acid encoding the human ChAT may have a fragment of the sequence (e.g., less than 50% of the sequence) set forth in SEQ ID NO:2 above, and may encode any of the ChAT polypeptides as discussed above.

The AAV sequences of the vector typically include the cis-acting 5′ and 3′ inverted terminal repeat sequences. The ITR sequences are about 145 bp in length. An example of such a molecule is a “cis-acting” plasmid containing the nucleic acid sequence encoding a ChAT, in which the nucleic acid sequence encoding a ChAT and associated regulatory elements are flanked by the 5′ and 3′ AAV ITR sequences. The AAV ITR sequences may be obtained from any known AAV, including presently identified mammalian AAV types. The 5′ and 3′ AAV ITR sequences may be AAV9 5′ and 3′ ITR sequences, respectively, or AAV2 5′ and 3′ ITR sequences, respectively, or AAV3 5′ and 3′ ITR sequences, respectively. In some embodiments, the nucleic acid sequence encoding ChAT may be packaged as a single stranded (ss) nucleotide sequence.

In some embodiments, the AAV sequences of the vector include the 5′ and 3′ AAV2 ITR sequences, respectively. In some cases, the nucleic acid present in the rAAV (i.e., the nucleic acid encoding ChAT) may encode the 5′ AAV2 ITR sequence that is at least 75%, 80%, 85%, 90%, 95%, 96%, 97%, 98%, or 99% or a 100% identical to the nucleic acid sequence:

(SEQ ID NO: 3)
CCTGCAGGCAGCTGCGCGCTCGCTCGCTCACTGAGGCCGCCCGGGCAAA
GCCCGGGCGTCGGGCGACCTTTGGTCGCCCGGCCTCAGTGAGCGAGCGA
GCGCGCAGAGAGGGAGTGGCCAACTCCATCACTAGGGGTTCCT.

In these instances, the nucleic acid present in the rAAV may further encode the 3′ AAV2 ITR sequence that is at least 75%, 80%, 85%, 90%, 95%, 96%, 97%, 98%, or 99% or a 100% identical to the nucleic acid sequence:

(SEQ ID NO: 4)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGCTGCCTGCAGG.

In some embodiments, the nucleic acid present in the rAAV (i.e., the nucleic acid encoding ChAT) is a single-stranded AAV (ssAAV) vector. In other embodiments, the nucleic acid sequence encoding ChAT further encodes an inverted self-complementary repeat of the ChAT such that an inverted self-complementary repeat of CHAT may fold into double stranded (ds) DNA without the requirement for DNA synthesis or base-pairing between multiple vector genomes (i.e., the nucleic acid present in the rAAV is a self-complementary AAV [scAAV] vector). The coding region of the scAAV vector forms an intramolecular double-stranded DNA template in contrast to a standard AAV vector having a single-stranded DNA template. The two complementary sequences of the scAAV coding region associate to form dsDNA that is ready for expression. An scAAV vector does not require cell mediated synthesis of a second strand like the standard AAV vector. Consequently, the scAAV vector can provide greater and longer expression of the transgene that it carries relative to the standard AAV vector. Whereas an AAV carrying an AAV vector can be efficiently produced with a standard AAV vector carrying a transgene having a total length of about 4 kb to about 5 kb, the transgene in an scAAV vector has a total length of about 2 kb to about 3 kb.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus or virion produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art.

In certain aspects, the nucleic acid present in the rAAV (i.e., the nucleic acid encoding ChAT) includes one or more polyA signals. PolyA signal sequences typically include a short sequence that triggers polyadenylation of an mRNA and, e.g., can include additional sequences surrounding a shorter sequence such that RNA stability, expression, and/or function can be enhanced. Various polyA signal sequences may be used for the coding sequences of various embodiments of the nucleic acid encoding ChAT. In certain embodiments, the nucleic acid present in the rAAV may further encode the poly A signal sequence that is at least 75%, 80%, 85%, 90%, 95%, 96%, 97%, 98%, or 99% or a 100% identical to the nucleic acid sequence of SEQ ID NO:5 (hGH polyA). In certain embodiments, the nucleic acid present in the rAAV may further encode the polyA signal sequence that is at least 75%, 80%, 85%, 90%, 95%, 96%, 97%, 98%, or 99% or a 100% identical to the nucleic acid sequence of SEQ ID NO:6 (SV40 polyA). In certain embodiments, the nucleic acid present in the rAAV may further encode the polyA signal sequence that is at least 75%, 80%, 85%, 90%, 95%, 96%, 97%, 98%, or 99% or a 100% identical to the nucleic acid sequence of SEQ ID NO:7 (bGH polyA). In certain embodiments, the nucleic acid present in the rAAV may further encode the polyA signal sequence that is at least 75%, 80%, 85%, 90%, 95%, 96%, 97%, 98%, or 99% or a 100% identical to the nucleic acid sequence of SEQ ID NO:8 (rbGlob polyA).

In certain aspects, the promoter may be a CMV, a chicken beta actin promoter, human β-actin/CMV hybrid promoter, chicken β-actin/CMV hybrid promoter, CMV-actin-globin (CAG) hybrid promoter, a CNS specific promoter, endogenous CHAT promoter for human ChAT, Hb9 enhancer composed of two minimal Hb9 promoter sequences (Hb9e), Hb9 enhancer fused to CMV minimal promoter (Hb9cmv), SYN1, Nppa, My12, and the like. In some cases, the promoter exhibits strong, ubiquitous, long-term expression in the central nervous system (CNS) of the subject (such as, e.g., exclusively to neurons). In these cases, the promoter may be a CBA hybrid intron (CBh) promoter or a SYN1 promoter. In certain aspects, the transgene (e.g., the human ChAT) promoter may be the CBh promoter and may comprise the following sequence or may comprise a sequence at least 85% identical (e.g., at least 90%, at least 95%, at least 97%, at least 98%, or at least 99% identical) to the following nucleic acid sequence:

(SEQ ID NO: 9)
CGTTACATAACTTACGGTAAATGGCCCGCCTGGCTGACCGCCCAACGACCCC
CGCCCATTGACGTCAATAGTAACGCCAATAGGGACTTTCCATTGACGTCAATGGGTG
GAGTATTTACGGTAAACTGCCCACTTGGCAGTACATCAAGTGTATCATATGCCAAGT
ACGCCCCCTATTGACGTCAATGACGGTAAATGGCCCGCCTGGCATTGTGCCCAGTAC
ATGACCTTATGGGACTTTCCTACTTGGCAGTACATCTACGTATTAGTCATCGCTATTA
CCATGGTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGCGGGG
GGGGGGGGGGGGCGCGCGCCAGGCGGGGGGGGGGGGCGAGGGGCGGGGGGGG
GCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGG
CGGGAGTCGCTGCGCGCTGCCTTCGCCCCGTGCCCCGCTCCGCCGCCGCCTCGCGCC
GCCCGCCCCGGCTCTGACTGACCGCGTTACTCCCACAGGTGAGCGGGCGGGACGGC
CCTTCTCCTCCGGGCTGTAATTAGCTGAGCAAGAGGTAAGGGTTTAAGGGATGGTTG
GTTGGTGGGGTATTAATGTTTAATTACCTGGAGCACCTGCCTGAAATCACTTTTTTTC
AG.

In certain aspects, the transgene (e.g., the human ChAT) promoter may be the SYN1 promoter and may comprise the following sequence or may comprise a sequence at least 85% identical (e.g., at least 90%, at least 95%, at least 97%, at least 98%, or at least 99% identical) to the following nucleic acid sequence:

(SEQ ID NO: 10)
GAGGGCCCTGCGTATGAGTGCAAGTGGGTTTTAGGACCAGGATGAGGCG
GGGTGGGGGTGCCTACCTGACGACCGACCCCGACCCACTGGACAAGCAC
CCAACCCCCATTCCCCAAATTGCGCATCCCCTATCAGAGAGGGGGAGGG
GAAACAGGATGCGGCGAGGCGCGTGCGCACTGCCAGCTTCAGCACCGCG
GACAGTGCCTTCGCCCCCGCCTGGCGGCGCGCGCCACCGCCGCCTCAGC
ACTGAAGGCGCGCTGACGTCACTCGCCGGTCCCCCGCAAACTCCCCTTC
CCGGCCACCTTGGTCGCGTCCGCGCCGCCGCCGGCCCAGCCGGACCGCA
CCACGCGAGGCGCGAGATAGGGGGGCACGGGCGCGACCATCTGCGCTGC
GGCGCCGGCGACTCAGCGCTGCCTCAGTCTGCGGTGGGCAGCGGAGGAG
TCGTGTCGTGCCTGAGAGCGCAG.

In some embodiments, the nucleic acid present in the rAAV (i.e., the nucleic acid encoding ChAT) used for gene therapy encodes, from 5′ to 3′, the CBh promoter, a human ChAT polypeptide, and the hGH polyA signal sequence. In these instances, the CBh promoter may comprise a sequence at least 75%, 80%, 85%, 90%, 95%, 96%, 97%, 98%, or 99% or a 100% identical to the nucleic acid sequence of SEQ ID NO:9, the human ChAT polypeptide may comprise a sequence at least 75%, 80%, 85%, 90%, 95%, 96%, 97%, 98%, or 99% or a 100% identical to the nucleic acid sequence of SEQ ID NO:2, and the hGH polyA signal sequence may comprise a sequence at least 75%, 80%, 85%, 90%, 95%, 96%, 97%, 98%, or 99% or a 100% identical to the nucleic acid sequence of SEQ ID NO:5.

In some embodiments, the nucleic acid present in the rAAV (i.e., the nucleic acid encoding ChAT) includes the CBh promoter, the 5′ and 3′ AAV2 ITR sequences, and the hGH polyA signal sequence. In these instances, the nucleic acid encoding ChAT may comprise the following sequence or may comprise a sequence having at least 70%, at least 75%, at least 80%, at least 85%, at least 90%, at least 95%, at least 97%, at least 98%, or at least 99% sequence identity to the following nucleic acid sequence:

(SEQ ID NO: 11)
cctgcaggcagctgcgcgctcgctcgctcactgaggccgcccgggcaaagcccgggcgtcgggcgacctttggtcgcccgg
cctcagtgagcgagcgagcgcgcagagagggagtggccaactccatcactaggggttcctCGTTACATAACTTACGGT
AAATGGCCCGCCTGGCTGACCGCCCAACGACCCCCGCCCATTGACGTCAATAGTAA
CGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATG
ACGGTAAATGGCCCGCCTGGCATTGTGCCCAGTACATGACCTTATGGGACTTTCCTA
CTTGGCAGTACATCTACGTATTAGTCATCGCTATTACCATGGTCGAGGTGAGCCCCA
CGTTCTGCTTCACTCTCCCCATCTCCCCCCCCTCCCCACCCCCAATTTTGTATTTATTT
ATTTTTTAATTATTTTGTGCAGCGATGGGGGCGGGGGGGGGGGGGGGGCGCGCGCC
AGGCGGGGCGGGGCGGGGCGAGGGGGGGGCGGGGCGAGGCGGAGAGGTGCGGC
GGCAGCCAATCAGAGCGGCGCGCTCCGAAAGTTTCCTTTTATGGCGAGGCGGCGGC
GGCGGCGGCCCTATAAAAAGCGAAGCGCGCGGCGGGCGGGAGTCGCTGCGCGCTGC
CTTCGCCCCGTGCCCCGCTCCGCCGCCGCCTCGCGCCGCCCGCCCCGGCTCTGACTG
ACCGCGTTACTCCCACAGGTGAGCGGGGGGACGGCCCTTCTCCTCCGGGCTGTAAT
TAGCTGAGCAAGAGGTAAGGGTTTAAGGGATGGTTGGTTGGTGGGGTATTAATGTTT
AATTACCTGGAGCACCTGCCTGAAATCACTTTTTTTCAGgccaccATGGCAGCAAAAAC
TCCCAGCAGTGAGGAGTCTGGGCTGCCCAAACTGCCCGTGCCCCCGCTGCAGCAGA
CCCTGGCCACGTACCTGCAGTGCATGCGACACTTGGTGTCTGAGGAGCAGTTCAGGA
AGAGCCAGGCCATTGTGCAGCAGTTTGGGGCCCCTGGTGGCCTCGGCGAGACCCTG
CAGCAGAAACTCCTGGAGCGGCAGGAGAAGACAGCCAACTGGGTGTCTGAGTACTG
GCTGAATGACATGTATCTCAACAACCGCCTGGCCCTGCCTGTCAACTCCAGCCCTGC
CGTGATCTTTGCTCGGCAGCACTTCCCTGGCACCGATGACCAGCTGAGGTTTGCAGC
CAGCCTCATCTCTGGTGTACTCAGCTACAAGGCCCTGCTGGACAGCCACTCCATTCC
CACTGACTGTGCCAAAGGCCAGCTGTCAGGGCAGCCCCTTTGCATGAAGCAATACT
ATGGGCTCTTCTCCTCCTACCGGCTCCCCGGCCATACCCAGGACACGCTGGTGGCTC
AGAACAGCAGCATCATGCCGGAGCCTGAGCACGTCATCGTAGCCTGCTGCAATCAG
TTCTTTGTCTTGGATGTTGTCATTAATTTCCGCCGTCTCAGTGAGGGGGATCTGTTCA
CTCAGTTGAGAAAGATAGTCAAAATGGCTTCCAACGAGGACGAGCGTTTGCCTCCA
ATTGGCCTGCTGACGTCTGACGGGAGGAGCGAGTGGGCCGAGGCCAGGACGGTCCT
CGTGAAAGACTCCACCAACCGGGACTCGCTGGACATGATTGAGCGCTGCATCTGCCT
TGTATGCCTGGACGCGCCAGGAGGCGTGGAGCTCAGCGACACCCACAGGGCACTCC
AGCTCCTTCACGGCGGAGGCTACAGCAAGAACGGGGCCAATCGCTGGTACGACAAG
TCCCTGCAGTTTGTGGTGGGCCGAGACGGCACCTGCGGTGTGGTGTGCGAACACTCC
CCATTCGATGGCATCGTCCTGGTGCAGTGCACTGAGCATCTGCTCAAGCACATGACG
CAGAGCAGCAGGAAGCTGATCCGAGCAGACTCCGTCAGCGAGCTCCCCGCCCCCCG
GAGGCTGCGGTGGAAATGCTCCCCGGAAATTCAAGGCCACTTAGCCTCCTCGGCAG
AAAAACTTCAACGAATAGTAAAGAACCTTGACTTCATTGTCTATAAGTTTGACAACT
ATGGGAAAACATTCATTAAGAAGCAGAAATGCAGCCCTGATGCCTTCATCCAGGTG
GCCCTCCAGCTGGCCTTCTACAGGCTCCATCGAAGACTGGTGCCCACCTACGAGAGC
GCGTCCATCCGCCGATTCCAGGAGGGACGCGTGGACAACATCAGATCGGCCACTCC
AGAGGCACTGGCTTTTGTGAGAGCCGTGACTGACCACAAGGCTGCTGTGCCAGCTTC
TGAGAAGCTTCTGCTCCTGAAGGATGCCATCCGTGCCCAGACTGCATACACAGTCAT
GGCCATAACAGGGATGGCCATTGACAACCACCTGCTGGCACTGCGGGAGCTGGCCC
GGGCCATGTGCAAGGAGCTGCCCGAGATGTTCATGGATGAAACCTACCTGATGAGC
AACCGGTTTGTCCTCTCCACTAGCCAGGTGCCCACAACCACGGAGATGTTCTGCTGC
TATGGTCCTGTGGTCCCAAATGGGTATGGTGCCTGCTACAACCCCCAGCCAGAGACC
ATCCTTTTCTGCATCTCTAGCTTTCACAGCTGCAAAGAGACTTCTTCTAGCAAGTTTG
CAAAAGCTGTGGAAGAAAGCCTCATTGACATGAGAGACCTCTGCAGTCTGCTGCCG
CCTACTGAGAGCAAGCCATTGGCAACAAAGGAAAAAGCCACGAGGCCCAGCCAGG
GACACCAACCTTGAaggaacccctagtgatggagttggccactccctctctgcgcgctcgctcgctcactgaggccgggcga
ccaaaggtcgcccgacgcccgggctttgcccgggcggcctcagtgagcgagcgagcgcgcagctgcctgcagg.

In some embodiments, the sequence encoding the hGH polyA signal sequence is replaced with, or included in addition to, a sequence encoding SV40 polyA, bGH polyA and/or rbGlob polyA. In some embodiments, the opal stop codon (TGA) is replaced with an amber stop codon (TAG) or an ochre stop codon (TAA). In some embodiments, the Kozak sequence is omitted or may be engineered for enhanced transgene expression according to any known biomolecular engineering technique.

In some embodiments, the nucleic acid present in the rAAV (i.e., the nucleic acid encoding ChAT) used for gene therapy encodes, from 5′ to 3′, the SYN1 promoter, a human ChAT polypeptide, and the hGH polyA signal sequence. In these instances, the SYN1 promoter may comprise a sequence at least 75%, 80%, 85%, 90%, 95%, 96%, 97%, 98%, or 99% or a 100% identical to the nucleic acid sequence of SEQ ID NO: 10, the human ChAT polypeptide may comprise a sequence at least 75%, 80%, 85%, 90%, 95%, 96%, 97%, 98%, or 99% or a 100% identical to the nucleic acid sequence of SEQ ID NO:2, and the hGH polyA signal sequence may comprise a sequence at least 75%, 80%, 85%, 90%, 95%, 96%, 97%, 98%, or 99% or a 100% identical to the nucleic acid sequence of SEQ ID NO:5.

In some embodiments, the nucleic acid present in the rAAV (i.e., the nucleic acid encoding ChAT) includes the SYN1 promoter, the 5′ and 3′ AAV2 ITR sequences, and the hGH polyA signal sequence. In these instances, the nucleic acid encoding ChAT may comprise the following sequence or may comprise a sequence having at least 70%, at least 75%, at least 80%, at least 85%, at least 90%, at least 95%, at least 97%, at least 98%, or at least 99% sequence identity to the following nucleic acid sequence:

(SEQ ID NO: 12)
cctgcaggcagctgcgcgctcgctcgctcactgaggccgcccgggcaaagcccgggcgtcgggcgacctttggtcgcccgg
cctcagtgagcgagcgagcgcgcagagagggagtggccaactccatcactaggggttcctGAGGGCCCTGCGTATGAG
TGCAAGTGGGTTTTAGGACCAGGATGAGGCGGGGTGGGGGTGCCTACCTGACGACC
GACCCCGACCCACTGGACAAGCACCCAACCCCCATTCCCCAAATTGCGCATCCCCTA
TCAGAGAGGGGGAGGGGAAACAGGATGCGGCGAGGCGCGTGCGCACTGCCAGCTT
CAGCACCGCGGACAGTGCCTTCGCCCCCGCCTGGCGGCGCGCGCCACCGCCGCCTC
AGCACTGAAGGCGCGCTGACGTCACTCGCCGGTCCCCCGCAAACTCCCCTTCCCGGC
CACCTTGGTCGCGTCCGCGCCGCCGCCGGCCCAGCCGGACCGCACCACGCGAGGCG
CGAGATAGGGGGGCACGGGCGCGACCATCTGCGCTGCGGCGCCGGCGACTCAGCGC
TGCCTCAGTCTGCGGTGGGCAGCGGAGGAGTCGTGTCGTGCCTGAGAGCGCAGgccac
CATGGCAGCAAAAACTCCCAGCAGTGAGGAGTCTGGGCTGCCCAAACTGCCCGTGC
CCCCGCTGCAGCAGACCCTGGCCACGTACCTGCAGTGCATGCGACACTTGGTGTCTG
AGGAGCAGTTCAGGAAGAGCCAGGCCATTGTGCAGCAGTTTGGGGCCCCTGGTGGC
CTCGGCGAGACCCTGCAGCAGAAACTCCTGGAGCGGCAGGAGAAGACAGCCAACTG
GGTGTCTGAGTACTGGCTGAATGACATGTATCTCAACAACCGCCTGGCCCTGCCTGT
CAACTCCAGCCCTGCCGTGATCTTTGCTCGGCAGCACTTCCCTGGCACCGATGACCA
GCTGAGGTTTGCAGCCAGCCTCATCTCTGGTGTACTCAGCTACAAGGCCCTGCTGGA
CAGCCACTCCATTCCCACTGACTGTGCCAAAGGCCAGCTGTCAGGGCAGCCCCTTTG
CATGAAGCAATACTATGGGCTCTTCTCCTCCTACCGGCTCCCCGGCCATACCCAGGA
CACGCTGGTGGCTCAGAACAGCAGCATCATGCCGGAGCCTGAGCACGTCATCGTAG
CCTGCTGCAATCAGTTCTTTGTCTTGGATGTTGTCATTAATTTCCGCCGTCTCAGTGA
GGGGGATCTGTTCACTCAGTTGAGAAAGATAGTCAAAATGGCTTCCAACGAGGACG
AGCGTTTGCCTCCAATTGGCCTGCTGACGTCTGACGGGAGGAGCGAGTGGGCCGAG
GCCAGGACGGTCCTCGTGAAAGACTCCACCAACCGGGACTCGCTGGACATGATTGA
GCGCTGCATCTGCCTTGTATGCCTGGACGCGCCAGGAGGCGTGGAGCTCAGCGACA
CCCACAGGGCACTCCAGCTCCTTCACGGCGGAGGCTACAGCAAGAACGGGGCCAAT
CGCTGGTACGACAAGTCCCTGCAGTTTGTGGTGGGCCGAGACGGCACCTGCGGTGTG
GTGTGCGAACACTCCCCATTCGATGGCATCGTCCTGGTGCAGTGCACTGAGCATCTG
CTCAAGCACATGACGCAGAGCAGCAGGAAGCTGATCCGAGCAGACTCCGTCAGCGA
GCTCCCCGCCCCCCGGAGGCTGCGGTGGAAATGCTCCCCGGAAATTCAAGGCCACTT
AGCCTCCTCGGCAGAAAAACTTCAACGAATAGTAAAGAACCTTGACTTCATTGTCTA
TAAGTTTGACAACTATGGGAAAACATTCATTAAGAAGCAGAAATGCAGCCCTGATG
CCTTCATCCAGGTGGCCCTCCAGCTGGCCTTCTACAGGCTCCATCGAAGACTGGTGC
CCACCTACGAGAGCGCGTCCATCCGCCGATTCCAGGAGGGACGCGTGGACAACATC
AGATCGGCCACTCCAGAGGCACTGGCTTTTGTGAGAGCCGTGACTGACCACAAGGC
TGCTGTGCCAGCTTCTGAGAAGCTTCTGCTCCTGAAGGATGCCATCCGTGCCCAGAC
TGCATACACAGTCATGGCCATAACAGGGATGGCCATTGACAACCACCTGCTGGCAC
TGCGGGAGCTGGCCCGGGCCATGTGCAAGGAGCTGCCCGAGATGTTCATGGATGAA
ACCTACCTGATGAGCAACCGGTTTGTCCTCTCCACTAGCCAGGTGCCCACAACCACG
GAGATGTTCTGCTGCTATGGTCCTGTGGTCCCAAATGGGTATGGTGCCTGCTACAAC
CCCCAGCCAGAGACCATCCTTTTCTGCATCTCTAGCTTTCACAGCTGCAAAGAGACT
TCTTCTAGCAAGTTTGCAAAAGCTGTGGAAGAAAGCCTCATTGACATGAGAGACCTC
TGCAGTCTGCTGCCGCCTACTGAGAGCAAGCCATTGGCAACAAAGGAAAAAGCCAC
GAGGCCCAGCCAGGGACACCAACCTTGAaggaacccctagtgatggagttggccactccctctctgcgcgctcg
ctcgctcactgaggccgggcgaccaaaggtcgcccgacgcccgggctttgcccgggcggcctcagtgagcgagcgagcgcgcagctg
cctgcagg.

In some embodiments, the sequence encoding the hGH polyA signal sequence is replaced with, or included in addition to, a sequence encoding SV40 polyA, bGH polyA and/or rbGlob polyA. In some embodiments, the opal stop codon (TGA) is replaced with an amber stop codon (TAG) or an ochre stop codon (TAA). In some embodiments, the Kozak sequence is omitted or may be engineered for enhanced transgene expression according to any known biomolecular engineering technique.

In certain aspects, the AAV vector is selected from the group consisting of AAV1, AAV2, AAV4, AAV5, AAV6, AAV8-PHP.eB, AAV9-PHP.eB AAV-retro, AAV-Rh10, AAV-PHP.B, AAV-PHP.eB, AAV-PHP.S or AAV-Anc80, AAV9, and variants thereof, e.g., variants of a parental AAV modified to increase CNS targeting by the AAV vector. In certain aspects, the AAV vector is AAV9.

In certain aspects, the type of AAV vector administered to the subject may depend on the tissue or body part targeted. For example, AAV capsids (such as, e.g., AAV9 and/or AAV-Rh10 capsids) that efficiently localize in the CNS and/or have efficient transduction in neuronal cells (e.g., cholinergic neurons), may be used to target the ChAT gene to the CNS of the subject. An AAV vector (i.e., rAAV virion) may be used to target any component of the CNS of the subject. As discussed above, the rAAV virion of the present disclosure may include a rAAV viral particle composed of at least one AAV capsid protein. In some cases, the rAAV viral particle is composed of all of the capsid proteins of a wild-type AAV such as, e.g., all of the capsid proteins of wild-type AAV9 or AAV-Rh10. In some cases, the rAAV viral particle is composed of capsid proteins from multiple wild-type AAVs. In some cases, the rAAV viral particle is composed of at least one AAV capsid protein that has been engineered to have decreased immunoreactivity in the subject and/or enhanced tropism (i.e., enhanced localization and/or enhanced transduction) in the CNS of the subject such as, e.g., cholinergic neurons of the subject's CNS. The AAV capsid protein may be engineered using any known protein engineering technique including, but not limited to, those discussed above. In these cases, the rAAV viral particle may be composed of capsid proteins from multiple wild-type or engineered AAVs or may be composed of all of the capsid proteins of an engineered AAV.

Amino acid sequences for suitable AAV capsid proteins of the administered rAAV virion can be found in a variety of databases such as the UniProt database and/or the NCBI database. It is well known to those skilled in the art that a variety of substitutions and alterations can be made to the amino acid sequence of a polypeptide with little to no change in the function of the polypeptide such as, e.g., conservative substitutions and/or substitutions a distance away from functional or active sites. The rAAV virion administered to the subject may include AAV capsid proteins and AAV plasmid components of the same or differing serotypes. For example, the rAAV virion may include all of the capsid proteins of a wild-type or engineered AAV9 and ITRs from AAV9, or AAV2 or AAV3. ITRs may be wild type or may be engineered for enhanced transgene expression according to any known biomolecular engineering technique. Suitable ITRs can be found in a variety of databases such as the UniProt database and/or the NCBI database.

### Recombinant AAV Virion Administration Methods

The rAAV virion may be delivered to a subject in compositions according to any appropriate methods known in the art. The rAAV virion, preferably suspended in a physiologically compatible carrier or a pharmaceutically acceptable excipient (i.e., in a composition), may be administered to a subject, such as, for example, a human, mouse, rat, cat, dog, sheep, rabbit, horse, cow, goat, pig, guinea pig, hamster, chicken, turkey, or a non-human primate (e.g., Macaque).

The rAAV virions are administered in sufficient amounts to transduce the cells of a desired tissue and to provide sufficient levels of gene transfer and expression without undue adverse effects. Conventional and pharmaceutically acceptable routes of administration include, but are not limited to, direct delivery to the selected organ, system, or body part of the subject (e.g., direct delivery to the CNS of the subject), intraocular, intravenous, intramuscular, subcutaneous, intradermal, intrathecal, intraventricular, intracranial and other parental routes of administration. In certain circumstances it will be desirable to deliver the rAAV-based therapeutic constructs in suitably formulated pharmaceutical compositions disclosed herein either intravenously, intrathecally, intraventricularly, or intracranially. In certain aspects the administration may be into the brain such as, e.g., intraparenchymally, intracerebroventricularly, into the cisterna magna or subpial.

Delivery of the virions to a mammalian subject may be by direct administration to the nervous system of the subject such as the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord, cranium, spinal canal, subarachnoid space and neuromuscular junction, or cerebellar lobule, with a needle, catheter or related device, using neurosurgical or injection techniques known in the art, such as by stereotactic injection.

Moreover, in certain instances, delivery of the rAAV virions to a mammalian subject may be by intravenous injection. In some embodiments, the mode of administration of rAAV virions is by portal vein injection. Administration into the bloodstream may be by injection into a vein, an artery, or any other vascular conduit. In some embodiments, administration of rAAV virions into the bloodstream is by way of isolated limb perfusion, a technique well known in the surgical arts, the method essentially enabling the artisan to isolate a limb from the systemic circulation prior to administration of the rAAV virions. Routes of administration may be combined, if desired.

The compositions of the invention may include a rAAV alone, or in combination with one or more other viruses (e.g., a second rAAV encoding one or more different transgenes). In some embodiments, a composition includes 1, 2, 3, 4, 5, 6, 7, 8, 9, 10, or more different rAAVs each having one or more different transgenes. In certain aspects, the composition may include an additional therapeutic agent for treatment for an NMJ disorder such as CHAT-CMS.

In a further aspect, the present invention provides a viral vector or a virion of the present invention or a pharmaceutical composition of the present invention for use as a medicament.

In a further aspect, the present invention provides a viral vector or a virion of the present invention or a pharmaceutical composition of the present invention for use in a method of treating an NMJ disorder, preventing an NMJ disorder, or for increasing ChAT activity and/or ACh levels in a subject in need thereof. For example, the viral vector or virion may find use in treating CHAT-CMS or other diseases of the central and autonomic nervous systems with deficient cholinergic function such as Alzheimer Disease and Parkinson Disease.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Optionally, the compositions of the present disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art.

An effective amount of a rAAV is an amount sufficient to transduce a target animal, such as transduce a desired tissue of a target animal. In some embodiments, an effective amount of a rAAV is an amount sufficient to produce a stable somatic transgenic animal model. The effective amount will depend primarily on factors such as the species, age, weight, health of the subject, and the tissue to be targeted, and may thus vary among animals and tissue. For example, an effective amount of the rAAV is generally in the range of from about 1 ml to about 100 ml of solution containing from about 10⁹ to 10¹⁶ genome copies. In some cases, a dosage between about 10¹² to 10¹⁵ rAAV genome copies is appropriate. In certain preferred embodiments, 10¹² rAAV genome copies is effective to target brain, heart, liver, and pancreatic tissues. In certain embodiments, the dosage of rAAV is 10¹⁰, 10¹¹, 10¹², 10¹³, or 10¹⁴ genome copies per kg. In certain embodiments, the dosage of rAAV is 10¹⁰, 10¹¹, 10¹², 10¹³, 10¹⁴, or 10¹⁵ genome copies per subject. In some cases, stable transgenic animals are produced by multiple doses of a rAAV.

In certain aspects, an effective amount of a rAAV may be determined, at least in part, based on the toxicity of the rAAV in the subject. For example, a therapeutically effective dose may be under a certain level of genome copies associated with an enhanced risk level of liver toxicity, dorsal root ganglion (DRG) inflammatory reaction, transient thrombocytopenia and/or thrombotic microangiopathy. In some cases, the dose does not exceed 2×10¹⁴ vector genomes per kilogram in order to prevent, e.g., liver toxicity.

In certain aspects, a therapeutically effective dose provides a level and/or activity of ChAT that is at least 50% of the normal ChAT activity level and may result in a 50% or higher increase in the of ACh levels in the CNS of the subject, such as the NMJs of the subject, as compared to prior to the treatment. ChAT activity may be measured in a sample from the subject, such as, a blood sample or a fraction thereof (e.g., serum or plasma), cerebrospinal fluid (CSF), lymphocytes or tissue. The measured level may be compared to a normal level to determine whether a subject has increased ChAT activity. The measured level may be compared to a threshold level that is lower than the normal level to determine whether a subject has increased ChAT activity. The normal and threshold ChAT activity levels may be determined based on ChAT activity measurement in age matched and/or gender matched healthy subjects.

In certain aspects, a therapeutically effective dose results in the prevention and/or reduction in severity of symptoms manifested in the subject such as reductions of apneic episodes or increased time-off mechanical ventilation. In certain aspects, a therapeutically effective dose results in an increase in the quality of life of the subject. For example, a therapeutically effective dose may result in the ability to sit independently, ability to overcome gravity with proximal muscles, ability to stand up and ambulate and independency, etc. In some cases, the increase in the quality of life is measured by or determined using a change in the Myasthenia Gravis Activities of Daily Living (MG-ADL) total score or the Quantitative Myasthenia Gravis (QMG) total score. A therapeutically effective dose may increase life span of the subject by at least 1 year or more, e.g., at least 5 years, 10 years, 15 years, or more as compared to subjects not receiving treatment. In some cases, a therapeutically effective dose may result in the subject having a normal life span or, e.g., a life span unaffected by the NMJ disorder that they have (e.g., CHAT-CMS).

In some embodiments, rAAV compositions are formulated to reduce aggregation of AAV particles in the composition, particularly where high rAAV concentrations are present (e.g., ~10¹³ GC/ml or more). Methods for reducing aggregation of rAAVs are well known in the art and include, for example, addition of surfactants, pH adjustment, salt concentration adjustment, etc.

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared in such a way that a suitable dosage will be obtained in any given unit dose of the compound. Factors such as solubility, bioavailability, biological half-life, route of administration, product shelf life, immunoreactivity, and/or toxicity as well as other pharmacological considerations will be contemplated by one skilled in the art of preparing such pharmaceutical formulations and, as such, a variety of dosages and treatment regimens may be desirable.

The pharmaceutical forms suitable for injectable use may include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous, intraperitoneal, intrathecal, intraventricular, and intracranial administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV virions in the required amount in the appropriate solvent with various of the other ingredients enumerated herein, as required, followed by filtered sterilization. The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

### Combination Therapy

In certain aspects, the subject having an NMJ disorder may receive an additional therapy for treating the NMJ disorder. The additional therapy may be administered prior to, simultaneously with, or after administration of the rAAV of the present disclosure. In cases where the NMJ disorder is CHAT-CMS, the additional therapy may include administration of an agent, e.g., a compound for treating CHAT-CMS. Such an agent may be an anticholinesterase medication such Pyridostigmine (Mestinon), sympathomimetic drugs, such as Ephedrine and Albuterol, potassium-channel blockers such as 3,4 diaminopyridine (DAP), also referred as Amifampridine, and the like.

In certain aspects, the subject having a deficiency of acetylcholine (ACh) or a reduced ability to respond to ACh in their central nervous system (CNS) may receive an additional therapy increasing ACh or ChAT activity.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A method for treating a neuromuscular junction (NMJ) disorder in a subject, the method comprising administering to the subject a therapeutically effective dose of a recombinant adeno-associated viral (rAAV) vector comprising a nucleic acid encoding choline acetyltransferase (ChAT), wherein administering the rAAV vector results in expression of the ChAT in the subject thereby treating the NMJ disorder in the subject.

2. The method of Aspect 1, wherein the NJM disorder is a congenital myasthenic syndrome (CMS) or myasthenia gravis (MG).

3. The method of Aspect 2, wherein the NJM disorder is a CMS.

4. The method of Aspect 3, wherein the CMS is a presynaptic CMS.

5. The method of Aspects 3 or 4, wherein the CMS is CMS with episodic apnea (CMS-EA).

6. The method of any one of Aspects 2 to 5, wherein the CMS is associated with a mutation in the endogenous CHAT gene of the subject.

7. The method of Aspect 6, wherein the mutation reduces the enzymatic activity of ChAT in the subject.

8. The method of Aspect 7, wherein the mutation is Val136Met, Arg186Trp, Val194Leu, Arg207His, Pro211Ala, Arg566Cys, and/or Ser694Cys, wherein the amino acid numbering is based on the amino acid sequence set forth in SEQ ID NO:1.

9. The method of Aspect 8, wherein the mutation is Val136Met and/or Arg207His.

10. The method of any of the preceding aspects, wherein the subject is eight years of age or younger and has a body weight sufficient to allow the administration of an effective dose of AAV safely.

11. The method of Aspect 10, wherein the subject is a fetus in utero or a newborn.

12. The method according to any of the preceding aspects, wherein the subject is a human and the ChAT is a human ChAT.

13. The method of any of the preceding aspects, wherein the rAAV virion comprises AAV-retro, AAV-Rh10, AAV-PHP.B, AAV-PHP.eB, AAV-PHP.S or AAV-Anc80 capsid proteins.

14. The method of any one of Aspects 1 to 12, wherein the rAAV virion comprises AAV9 capsid proteins.

15. The method of any of the preceding aspects, wherein the rAAV virion comprises a nucleic acid comprising an AAV inverted terminal repeat, a promoter operably linked to the nucleic acid encoding ChAT, and an AAV inverted terminal repeat.

16. The method of Aspect 15, wherein the promoter exhibits strong, ubiquitous, long-term expression in the central nervous system (CNS) of the subject.

17. The method of Aspect 16, wherein the promoter is a CBA hybrid intron (CBh) promoter.

18. The method of Aspects 15 or 17, wherein the nucleic acid encoding ChAT encodes a single stranded (ss) ChAT.

19. The method of Aspects 15 or 17, wherein the nucleic acid encoding ChAT further encodes an inverted self-complementary repeat of the ChAT.

20. The method of any of the preceding aspects, wherein the rAAV vector is AAV-2, AAV-retro, AAV-Rh10, AAV-PHP.B, AAV-PHP.eB, AAV-PHP.S, AAV-Anc80, AAV-9, or a vector derived therefrom.

21. The method of any one of Aspects 15 to 20, wherein the ITRs are AAV2 ITRs, AAV3 ITRs, or AAV9 ITRs, optionally, wherein the AAV ITRs are AAV2 ITRs, the promoter is a CAG promoter, a CMV promoter, a CBA promoter, or a CBh promoter and the rAAV comprises AAV9 capsid.

22. The method according to Aspect 15, wherein the rAAV virion comprises a nucleic acid comprising at least 70%, 75%, 80%, 85%, 90%, 95%, or 100% sequence identity to SEQ ID NO:11 or SEQ ID NO:12.

23. The method according to Aspect 22, wherein the rAAV virion comprises an AAV9 capsid.

24 The method of any of the preceding aspects, wherein the rAAV virion is administered by intravenous injection.

25. The method of any of the preceding aspects, wherein the rAAV virion is administered by direct injection into the CNS.

26. The method of Aspect 25, wherein the injection is an intrathecal, intraventricular, or intracranial injection.

27. The method of any of the preceding aspects, wherein the therapeutically effective dose of the rAAV virion comprises a dose of less than 2×10¹⁴ viral genomes per kilogram (vg/kg).

28. The method of Aspect 26 or 27, wherein the therapeutically effective dose of the rAAV virion comprises a dose of about 8×10¹², 2×10¹³, or 3.3×10¹³ viral genomes per kilogram (vg/kg).

29. The method of any of the preceding aspects, wherein the rAAV virion is administered with a pharmaceutically acceptable diluent, carrier or excipient.

30. The method of any of the preceding aspects, wherein the rAAV virion is administered to the subject on two or more occasions.

31. A recombinant adeno-associated viral (rAAV) virion comprising a nucleic acid encoding choline acetyltransferase (ChAT).

32. The rAAV virion of Aspect 31, wherein the rAAV virion comprises an rAAV vector comprising an AAV inverted terminal repeat, a promoter/enhancer, a nucleic acid sequence encoding choline acetyltransferase (ChAT), and an AAV inverted terminal repeat.

33. The rAAV virion of Aspect 32, wherein the ChAT is a human ChAT.

34. The rAAV virion of any one of Aspects 32 or 33, wherein the rAAV virion comprises AAV-retro, AAV-Rh10, AAV-PHP.B, AAV-PHP.eB, AAV-PHP.S or AAV-Anc80 capsid proteins.

35. The rAAV virion of any one of Aspects 32 or 33, wherein the rAAV virion comprises AAV9 capsid proteins.

36. The rAAV virion of any one of Aspects 32 to 35, wherein the rAAV vector is AAV-2, AAV-retro, AAV-Rh10, AAV-PHP.B, AAV-PHP.eB, AAV-PHP.S, AAV-Anc80, AAV-9, or a vector derived therefrom.

37. The rAAV virion of any one of Aspects 32 to 35, wherein the ITRs are AAV2 ITRs, AAV3 ITRs, or AAV9 ITRs, optionally, wherein the AAV ITRs are AAV2 ITRs, the promoter is a CAG promoter, a CMV promoter, a CBA promoter, or a CBh promoter and the rAAV comprises AAV9 capsid.

38. The rAAV virion of Aspect 33, wherein the rAAV virion comprises a nucleic acid comprising at least 70%, 75%, 80%, 85%, 90%, 95%, or 100% sequence identity to SEQ ID NO: 11 or SEQ ID NO:12.

39. The rAAV virion of Aspect 38, wherein the rAAV virion comprises AAV9 capsid proteins.

40. The rAAV virion of any one of Aspects 32 to 39, wherein the promoter exhibits strong, ubiquitous, long-term expression in the central nervous system (CNS) of the subject.

41. The rAAV virion of Aspect 40, wherein the promoter is a CBA hybrid intron (CBh) promoter.

42. The rAAV virion of any one of Aspects 33 to 41, wherein the nucleic acid sequence encoding ChAT produces a single stranded sequence encoding the ChAT.

43. The rAAV virion of any one of Aspects 33 to 41, wherein the nucleic acid sequence encoding ChAT further encodes an inverted self-complementary repeat of the ChAT.

44. A composition comprising the rAAV virion of any one of Aspects 33 to 43 and a pharmaceutically acceptable diluent, carrier, or excipient.

45. The composition of Aspect 44, wherein the composition comprises a therapeutically effective dose of the rAAV virion.

46. The composition of Aspect 45, wherein administration of the therapeutically effective dose results in an expression level of ChAT activity that is at least 50% of the normal ChAT activity level.

47. The composition of Aspect 46, wherein administration of the therapeutically effective dose results in a reduction of apneic episodes.

48. The composition of any one of Aspects 44 to 47, wherein the composition comprises about 2.4×10¹³, 6×10¹³, or 9.9×10¹³ viral genomes (vg) subject.

### EXAMPLES

As demonstrated in the above disclosure, the present invention has a wide variety of applications. The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g., amounts, concentrations, etc.) but some experimental errors and deviations should be accounted for.

### Example 1: CHAT Mutation Phenotype-Genotype Correlation

Clinical studies in large number of patients with CMS due to CHAT mutations have revealed a broad spectrum of clinical manifestations and severity of symptoms that ranged from infantile onset of mild weakness with episodic apnea to a neonatal onset of severe weakness, respiratory failure, and cognitive developmental delay [1]. More recent studies attempting to correlate the molecular changes caused by CHAT mutations with the kinetics of the enzyme and the clinical manifestations have revealed an association between the severity of symptoms with the proximity of the molecular changes to the catalytic pore of the enzyme [2] [3].

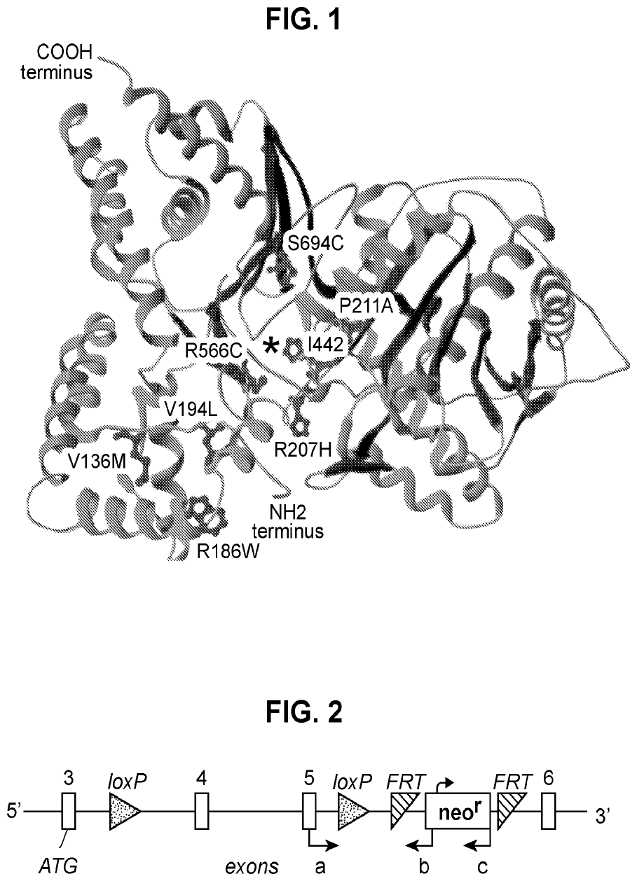

The genotype-phenotype correlation was investigated in seven families with affected members. It was found that the R207H and V136M mutations were associated with the most severe phenotypes (FIG. 1). The mutation p.Arg207His is located at the active-site tunnel while p.Val136Met, although distant from the active pore, is associated with poor ChAT expression in HEK cells [4].

FIG. 1 depicts the structure of human ChAT and identified mutations. The catalytic histidine is depicted in red, and the active-site tunnel is denoted with an asterisk. The structure is represented in a ribbon diagram with α-helices in blue, β-sheet in green and coils in orange. The mutated residues in magenta are located as follows: p. Val136Met at coil 1, p.Arg186Trp at α-helix 3, p. Val194Leu at α-helix 4, p.Arg207His at coil 5, p.Pro211Ala at coil 5, p.Arg566Cys at β-sheet 12 and p.Ser694Cys at β-sheet 16. The p.Arg207His mutation, located at the active-site tunnel, is associated with the most severe phenotype. FIG. 1 was modified from Hinderer et al. (2018) Hum Gene Ther., 3:285-298 [4]. FIG. 1 was modified from Arredondo et al. (2015) Hum Mutat. 2015 Scp; 36 (9): 881-93 [2].

### Example 2: Adeno-Associated Virus Type 9-Mediated Gene Therapy of Choline Acetyltransferase-Deficient Mice

### Overview

The enzyme choline acetyltransferase (ChAT) synthesizes acetylcholine from acetyl-CoA and choline at the neuromuscular junction and at the nerve terminals of cholinergic neurons. Mutations in the ChAT gene (CHAT) result in a presynaptic congenital myasthenic syndrome (CMS) that often associates with life-threatening episodes of apnea. Knockout mice for Chat (Chat^{-/-}) die at birth. To circumvent the lethality of this model, mutant mice possessing loxP sites flanking Chat exons 4 and 5 were crossed with mice that expressed Cre-ER^{T2}. Injection of tamoxifen (Tx) at postnatal (P) day 11 in these mice induced downregulation of Chat, autonomic failure, weakness, and death. However, a proportion of Chat^{flox/flox}-Cre-ER^{T2} mice receiving at birth an intracerebroventricular injection of 2·10¹³ vg/kg adeno associated virus type 9 (AAV9) carrying human CHAT (AAV9-CHAT) survived a subsequent Tx injection and lived to adulthood without showing signs of weakness. Likewise, injection of AA9-CHAT by intracisternal injection at P28 after the onset of weakness also resulted in survival to adulthood. The expression of Chat in spinal motor neurons of Chat^{flox/flox}-Cre-ER^{T2} mice injected with Tx was markedly reduced, but AAV-injected mice showed a robust recovery of ChAT expression, which was mainly translated by the human CHAT RNA. The biodistribution of the viral genome was widespread but maximal in the spinal cord and brain of AAV-injected mice. No significant histopathological changes were observed in the brain, liver, and heart of AAV-injected mice after 1 year follow-up. Thus, AAV9-mediated gene therapy may provide an effective and safe treatment for patients severely affected with CHAT-CMS.

### Results

Injection of Tx into Double Homozygous Chat^{flox/Flox}-Cre-ER^{t2} Mice Resulted in the Arrest of Growth, Progressive Weakness, and Death

To circumvent the perinatal mortality of the Char^{flox/flox}-Cre-b-actin mice, double homozygous mutants for Chat^{flox/flox} and Cre-ER^{T2} were used. These animals were normal at birth, developed normally and were fertile. However, if they were injected with two IP injections of the estrogen agonist Tx 40 mg/kg 6-8 h apart at postnatal day (P) 11, the development of these mice was arrested; they became progressively weak, lost weight, and with rare exceptions (<5%), all died. In addition, most mice developed fecal impactions presumably due to lack of gastrointestinal peristalsis.

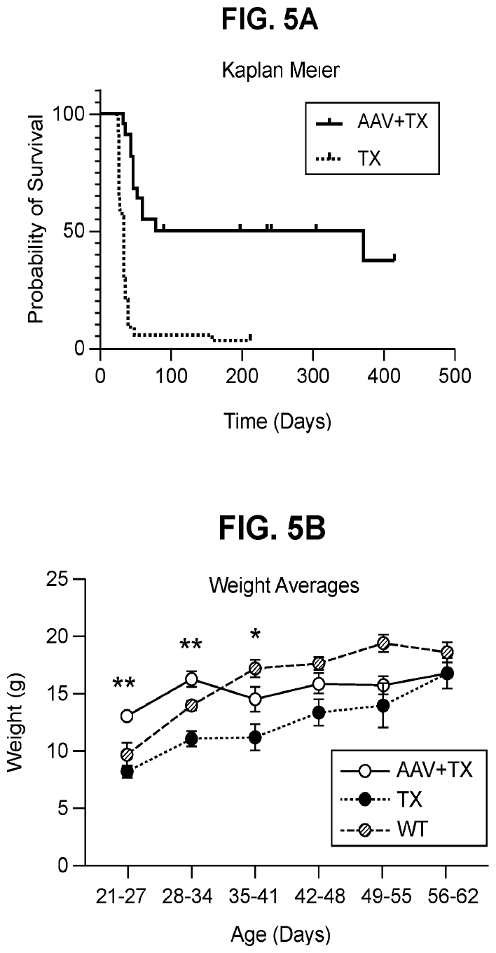

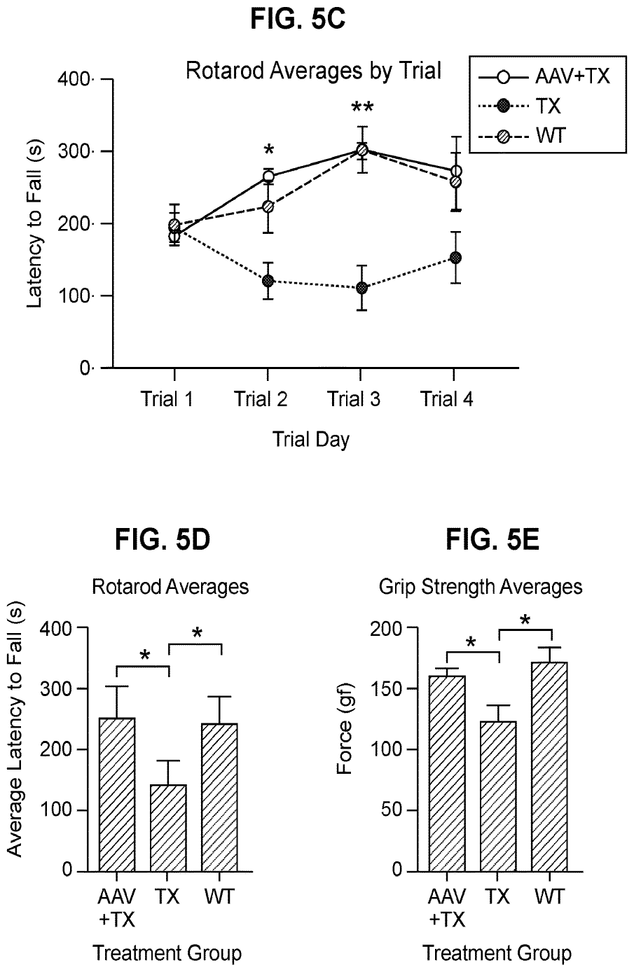

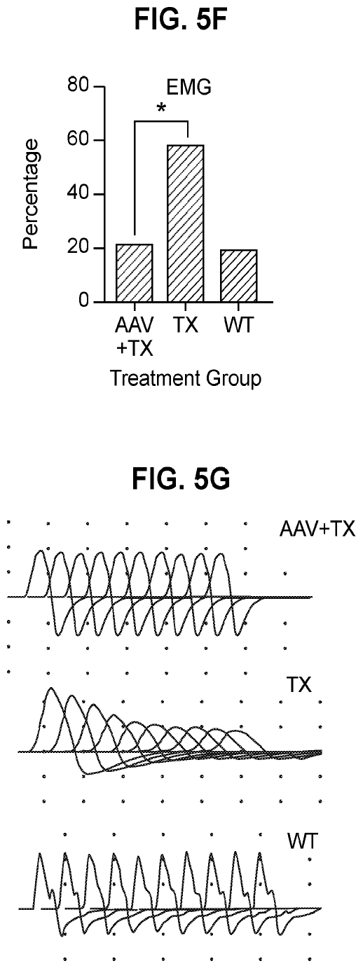

Close to half of the mice died before the end of the first month and most of the rest died by the end of the second month of life (FIG. 5A). Unexpectedly, despite the overall reduction of motor activity, injected mice occasionally made unusual high jumps, suggesting repression of an inhibitory neuronal circuitry in the spinal cord. WT mice (n=23) receiving similar IP injections of Tx at P11 as mutants developed no weakness and none died.

ICV Injection of AAV9-CHAT into Double Homozygous Chat^{flox/Flox}-Cre-ER^{T2} Mice at P1 Prevented Lethality and Prolonged Survival

After developing the model, the hypothesis of whether the survivability of Chat depleted mice could be improved by replenishing the ChAT enzyme using an AAV9 vector was first tested. It was found that about half of the mice injected with 2·10¹³ vg/kg of an AAV9-carrying human CHAT during the first 24 h after birth survived two subsequent IP injections of Tx 40 mg/kg 6 h apart at P11. Injected animals that survived showed no obvious difference with WT mice and if they were bred among themselves, they gave birth to normal double mutant pups that they were able to rear to maturity.

Moreover, even in mice injected with AAV9-CHAT that later died after the injection of Tx, the survival was longer than those injected with Tx alone as illustrated in the Kaplan-Meier analysis shown in FIG. 5A. Injection of the AAV9-CHAT at the dose of 5·10¹² vg/kg had no significant impact on survival (median survival of 37 days in comparison with 34 days in the Tx group). The weights of the AAV+Tx, Tx and WT groups were also measured throughout a 6-week period in 9 (AAV+Tx), 14 (Tx) and 8 (WT) mice at the starting time.

During the first 3 weeks the average weight of mice injected with AAV9-CHAT was higher than those injected with Tx alone, but there were no differences thereafter as shown in FIG. 5B. There was a mild reduction in the average weight of mice injected with AAV9-CHAT compared with WT, which was not significant at the end of the testing period.

Injection of AAV9-CHAT at Birth Improved the Motor Performance of Chat^{flox/Flox}-Cre-ER^{t2}

Next, the motor performance in three study groups: AAV9-CHAT plus Tx, Tx only, and WT mice was tested. Each of the three groups consisted of 5-week-old mice with the testing performed every week for 4 weeks. Beyond that point, the analysis was not continued as the number of surviving mice in Group 2 (Tx) was very small.

Rotarod: FIG. 5C shows that at week 1, the three tested groups did not perform significantly differently. However, as testing progressed, the AAV-treated mice and WT mice performed similarly, with a latency to fall ranging from 225 to 300 s, whereas Tx-treated mice fatigued more quickly, with a latency to fall ranging from 125 to 150 s. The overall performance of the Tx-only group was impaired in comparison with the other two groups (FIG. 5D).

Grip strength: The overall performance of Chat^{flox/flox}-Cre-ER^{T2} mice in the grip strength test was diminished in comparison with Tx-injected Chat^{flox/flox} Cre-ER^{T2} that had been previously injected with AAV9-CHAT and WT mice as shown in FIG. 5E.

EMG: Recordings were performed before and after a long train of stimuli at 30 Hz for 30 s. Decrements of compound muscle action potentials were observed in the three groups, especially with higher frequencies of stimulation, but were present more often in mice mutants injected only with Tx in comparison with mice injected with AAV vector and Tx as shown in FIGS. 5F and 5G.

FIGS. 5A to 5G demonstrates survival and motor performance of Chat^{flox/flox}-Cre-ER^{T2} mice treated and untreated with AAV9-CHAT. (FIG. 5A) The Kaplan-Meier analysis was used to assess mice survival. The analysis compared the survival of AAV9-CHAT treated (turquoise, n=22) and non-treated mice (red, n=34) enrolled during a period of 14 months. There were equal proportion of females and males in the two groups. Both groups received IP injections of Tx at P11. The tick marks on the curves represent censored mice euthanized for collection of tissue. Animals that had to be euthanized due to severe weakness were entered as death rather than as censored mice, regardless of whether tissue was collected or not. Note that most censored mice were long survivors in the AAV group, and these events were not counted for the estimation of the overall survival of the groups. About half of the mice treated with AAV+Tx lived until adulthood and showed no apparent weakness. In contrast, the mice only treated with Tx died or were euthanized due to severe weakness, much earlier and at a greater rate. The median survival in the AAV+Tx group was 224 days in comparison with 34 days in the Tx group. The differences between the two groups were analyzed with the log-rank test and found to be highly significant (p<0.001). (FIG. 5B) Weight curve showing reduction of the average weight in the Tx group in comparison with the AAV+Tx group (**p<0.0001, *p<0.01) during the first three weeks, but no differences between the groups afterward. (FIG. 5C) The rotarod experiments were conducted for four trial days in WT group (n=12), AAV9-CHAT+Tx group (n=9, 9, 9, and 7) and Tx-only group (n=14, 10, 5, and 3) and showed decreased average latency to fall in the Tx-only group in comparison with the other two groups at the second and third trials (p<0.05 and p<0.01, respectively). (FIG. 5D) The average latency to fall was decreased in the Tx-only group (p<0.01). (FIG. 5E) There was a reduction of the average grip strength in the Tx-only group in comparison with the AAV+Tx and WT groups (p<0.05). (FIG. 5F) Bar graph demonstrating higher percentage of mice showing decrement of CMAP amplitudes before and after continuous nerve stimulation for 30 s at 30 Hz in mice treated with Tx alone in comparison with mice treated with AAV+Tx (p<0.05, chi squared test). The data, which were not normally distributed, resulted from EMG studies performed in the Tx-only group (n=14), the AAV+Tx group (n=9), and the WT group (n=5). (FIG. 5G) Examples of repetitive nerve stimulation at 10 Hz in the three groups illustrating decrement of CMAP amplitudes in a mouse treated with Tx and absence of decrement in a WT mouse and a mouse treated with AAV+Tx.

IC/IP Injections of AAV9-CHAT into Double Homozygous Chat^{flox/Flox}-Cre-ER^{t2} Mice at P28 after the Onset of Weakness Prevented Weakness and Lethality

After confirming a beneficial effect of AAV9-CHAT injected at P1 it was asked whether a positive effect could be still observed if the vector was injected after the onset of weakness. To evaluate that, a dose of 2·10¹³ vg/kg of AAV9-CHAT was administered to P28 mice by an IC injection (101 L) with the remnant of the vector delivered intraperitoneally.

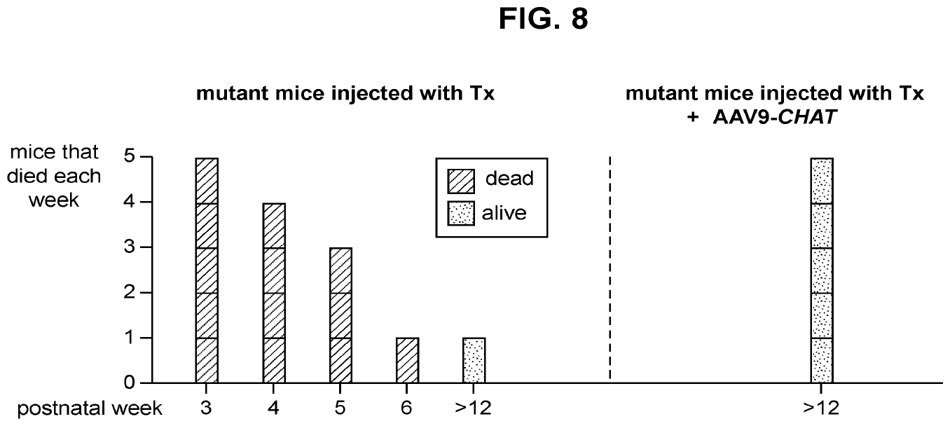

These experiments showed evidence that the AAV9-CHAT injected at P28 was efficacious to prevent motor deficit and lethality as five out of five mice injected with AAV9-CHAT survived for >12 weeks, whereas 13 of 14 non-injected mice died (FIG. 8). Rotarod test performed in four of the AAV-injected mice 1 year after the AAV injection revealed no significant differences with age matched WT mice.

FIG. 8 illustrates mortality of mutant mice injected and noninjected with AAV9-CHAT at P28 after the induction of disease.

A Single ICV Injection of AAV9-CHAT at Birth Increased the Expression of ChAT in the Spinal Motor Neurons of Chat^{flox/Flox}-Cre-ER^{t2}

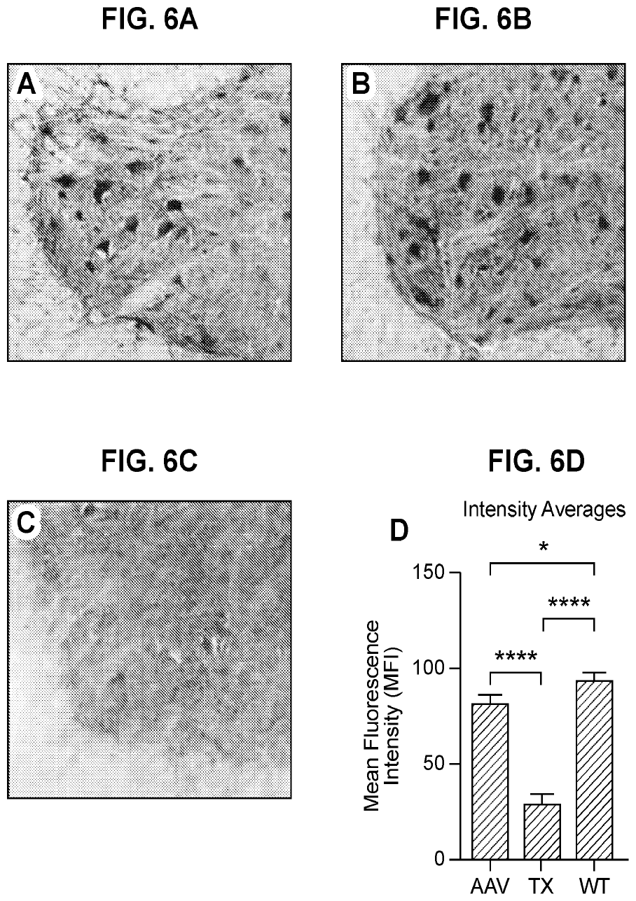

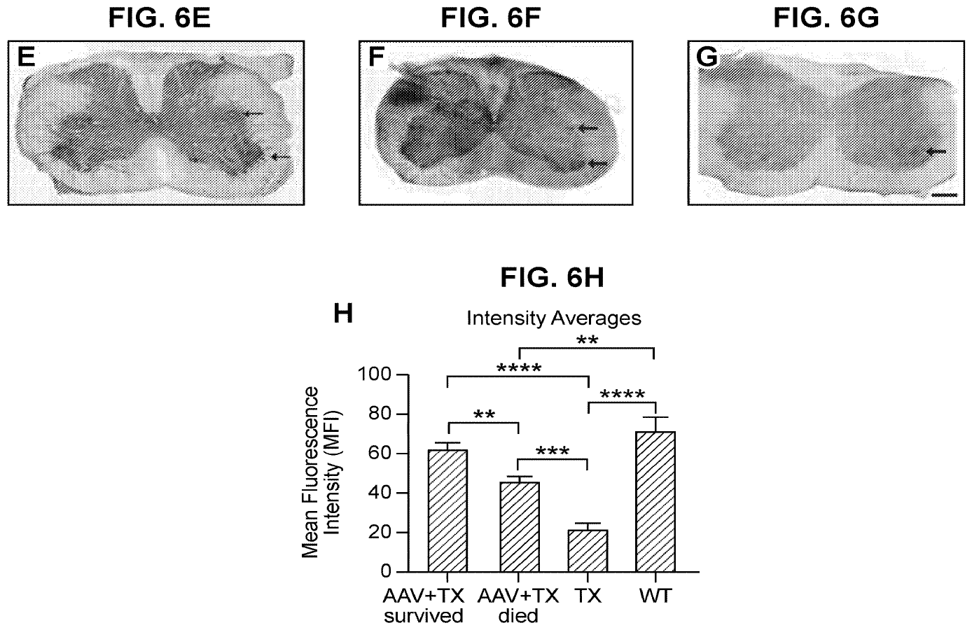

To evaluate the hypothesis that the weakness and lethality in the three study groups associated with diminished expression of the ChAT protein in the spinal cord the intensity of immunofluorescence (IF) of ChAT immunopositive neurons with cell body diameters >20 μm was quantified. These cells primarily populate the lateral aspect of the anterior horn (FIG. 6E). The analysis performed in sections of the lumbar spinal cord of WT (7), AAV (7), and Tx (3) groups showed that the IF intensity in spinal motor neurons (SMNs) of mice injected only with Tx was severely diminished in comparison with those from WT and mice treated with AAV9 and Tx (****p<0.0001).

The expression of ChAT in AAV-treated mice was only mildly diminished relative to that of WT mice (*p<0.05) (FIG. 6D). Similarly, the mean expression of Chat in the sections of cervical spine of mutant mice treated with Tx (5) was diminished in comparison with WT mice (21) and AAV-treated (33) mice (p<0.0001). In comparison with WT, there was reduction of ChAT IF in AAV-injected that died (p<0.01), but there was no significant reduction of ChAT IF in AAV-injected that survived long term (FIG. 6E, 6H).

Likewise, there was no significant difference between the percentage of lumbar SMNs with positive ChAT IF in AAV-treated mice that survived to adulthood (12.3-6.9, n=8) in comparison with WT (12.0-2.7, n=38). However, this was reduced in AAV-treated mice that were euthanized because of weakness (2.0-0.3, n=20), p<0.001.

FIGS. 6A to 6H depict expression of ChAT in SMNs of the lumbar and cervical spinal cord. (FIGS. 6A-6C) Examples of confocal microscope images of the lumbar spinal cord of a WT mouse (FIG. 6A), a mutant injected with AAV9-CHAT and Tx (FIG. 6B), and a mutant mouse injected only with Tx (FIG. 6C). There is strong expression of Chat in SMNs of the WT mouse and widespread expression of ChAT in SMNs of the mutant mouse treated with AAV9-CHAT and Tx. In contrast, there is severe reduction of Chat expression in the spinal cord of a mouse treated only with Tx. (FIG. 6D) Bar graph showing the average emission of Alexa Fluor 594 in SMNs of the anterior horn measuring >20 μm. The IF intensity in SMNs of mice injected only with Tx (red) was reduced in comparison with that of WT (blue) and that of mice treated with AAV9 and Tx (turquoise) (****p<0.0001). The expression of ChAT in AAV-treated mice was slightly diminished relative to that of WT mice (*p<0.05). (FIGS. 6E-6G) Examples of ChAT IF in the cervical spines of a WT (FIG. 6E), an AAV-treated (FIG. 6F), and a nontreated (FIG. 6G) mice. The white arrows point at SMNs of the anterior horn and intermediate column showing positive ChAT IF. (FIG. 6H) Bar graph showing the expression of ChAT in cervical spines of AAV-Tx mice in comparison with the Tx and WT groups (****p<0.0001; ***p<0.001; **p<0.01). The calibration mark represents 40 μm.

### Cell Transduction and Biodistribution of the AAV9-CHAT Vector

Since the beneficial effect of the AAV9-CHAT injection was sustained, it was next desired to assess the long-term persistence and biodistribution of the AAV9 vector in various tissues of three mice 1 year after the injection. The level of viral transduction was calculated using qPCR and primers designed to amplify the green fluorescence protein (GFP) gene. There was marked variability in the level of transduction of the viral genome.

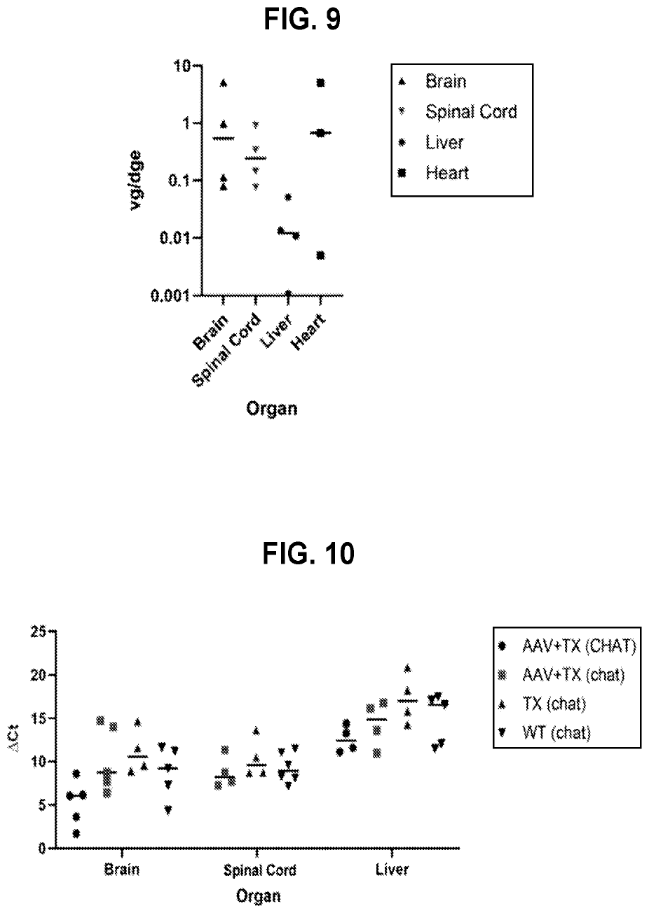

However, despite the exclusive administration of the vector to the CNS through an ICV injection, there was a widespread distribution of the viral genome inside and outside the CNS. Moreover, cells of the spinal cord showed as much transduction of the vector as the brain, but the transduction of the vector in the brain was higher than that in the liver (FIG. 9).

FIG. 9 provides a scatterplot showing great variability and widespread distribution of the viral genome inside and outside the CNS in Chat^{flox/flox}-Cre-ER^{T2} mice injected with AAV9-CHAT. Note that the level of transduction of the vector in the brain was higher than that in the liver (p<0.05).

### Expression of Human CHAT and Mouse Chat in the Brain and Liver

Given that the lethality of mice only injected with Tx associated with depletion of the Chat enzyme in the spinal cord, it was then asked whether low levels of Chat expression correlated with reduced levels of Chat. To determine that we performed RT-qPCR using RNA extracted from the brain, spinal cord, and liver, and primers designed to amplify human CHAT and mouse Chat. The relative levels of expression of both genes were calculated as ΔCt relative to the housekeeping gene GAPDH.

No difference was found between the level of Chat transcripts in the Tx group in comparison with that in the WT and AAV-injected mice, which is consistent with the notion that the impaired protein expression in the Tx group does not primarily result from reduced level of Chat transcripts, but from an impaired message in the transcripts (FIG. 10). Indeed, the Cre-mediated excision of exon 4-5, results in a frameshift, and an early termination codon predicting a truncated protein of <32 amino acids instead of the expected 641 amino acids.

Nevertheless, there was a trend toward a slightly less expression of Chat in the Tx group in comparison with WT in the tested tissues, which was not present in AAV-treated mice. This is likely due to a mild cross-reactivity between the primers used to amplify mouse Chat with human CHAT, but not between primers to amplify human CHAT with mouse Chat. In addition, it was found that in the brain, the level of expression of CHAT in AAV-treated mice was higher than that of Chat in AAV-treated mice and mice only treated with Tx. It was also higher than the level of expression of CHAT in the liver (p<0.01).

FIG. 10 illustrates gene expression in the brain, spinal cord, and liver of AAV-treated and untreated mice. A column scatter graph is provided showing that in the brain, there was maximal expression (lowest ΔCt) of CHAT in comparison with the other groups. In addition, in AAV-treated mice, there was increased expression of CHAT in the brain compared with that in the liver (Student's t-test, p<0.01). Although the levels of Chat transcripts were not significantly different among the three groups, a trend toward a slightly less expression of Chat in the Tx group in comparison with WT was present in all the sampled organs.

Histopathological Evaluation of CNS and Peripheral Organs after the Administration of AAV9-CHAT

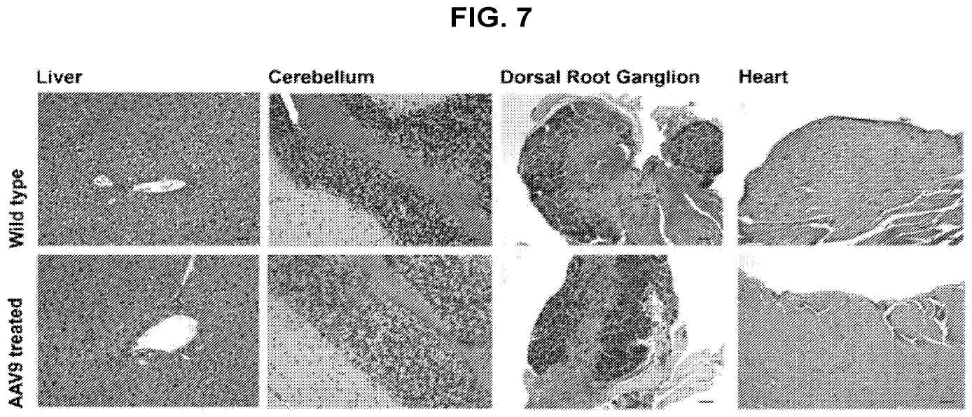

With promise of future use in humans, it was decided to assess the long-term effects of AAV9-CHAT treatment in mice. For that histological evaluation of the CNS, dorsal root ganglion, liver, heart, and gastrointestinal tract was performed with no significant treatment associated findings observed (FIG. 7). Mononuclear cellular infiltrates were seen in the liver of both treated and untreated mice, which were generally minimal to mild in severity and considered incidental and unrelated to AAV9 treatment. In one of the treated mice a benign liver adenoma was found, which was similarly considered incidental and not associated with AAV9 treatment.

FIG. 7 illustrates histological changes. Representative examples of histology sections of the liver, cerebellum, dorsal root ganglion, and heart from WT and AAV treated mice with no evidence of pathological changes. The calibration mark represents 20 μm.

### Summary

Because Cre-mediated excision of Chat exons 4 and 5 is not compatible with postnatal life, a conditional model induced by IP injection of Tx at P11 was used. In this model, several days after Tx injection, most mice developed a hunched back posture, tremors, weight loss, immobility, and death. In addition, the necropsy of these animals showed large accumulation of stools in the large intestine and fecal impactions. This likely results from the almost complete loss of Chat activity in enteric cholinergic neurons demonstrated in this same model when Chat loxP recombination was selectively induced in neural crest derived neurons.

Another unexpected feature was the occasional occurrence of a transient period of motor excitability in mice undergoing depletion of Chat. The simplest explanation for this finding is a reduction of Chat activity at recurrent branches of the SMNs contacting inhibitory Renshaw cells. This can conceivably result in decreased inhibition and paradoxical transient motor neuron excitation.

In this study, a single ICV injection of AAV9-CHAT at birth not only increased survival but also rescued about half of the injected mice with the lethal phenotype caused by the reduction of Chat at cholinergic synapses.

The efficiency of the ICV administration may be related to the enhanced accessibility of the transgene to SMNs when the vector is directly delivered into the CNS, but it may also reflect a more effective transduction of neurons at birth in comparison with adult life. This is explained by the pattern of transduction of AAV9, which is well-known to shift with age from a predominantly neuron to glial transduction. Nevertheless, the experiments involving the administration of the AAV9-CHAT at the fourth week well after the induction of weakness were still effective to prevent lethality.

In this investigation, the improved survival and motor performance of Chat^{flox/flox}-Cre-ER^{T2} injected with AAV-CHAT were associated with a commensurate increase of ChAT expression in the SMNs. However, a widespread transduction of CNS cells that do not naturally express Chat was of a particular concern. Although the rate limiting step in the resynthesis of ACh is the reuptake of choline through the CHT1, it has been reported that ChAT overexpression in neuronal cell lines can act as a transcription factor and enhance CHT1 expression.

Moreover, ChAT is also expressed in various types of non-neuronal cells, including epithelial, muscle, and immune cells, which suggests that ChAT participates in as yet unidentified functions. Thus, it is conceivable that the overexpression of ChAT may result in unknown and detrimental gain-of-functions processes. However, from the clinical standpoint, none of the mice treated with AAV-CHAT, even those surviving >1 year, developed dystonia, seizures, or tremors to suggest anomalous ACh production.

The RT-qPCR experiments suggested that the truncation of the protein resulting from the frameshift and early termination codon was the primary cause for the decreased expression of ChAT since the levels of Chat transcripts were not overtly reduced in the Tx-treated mutant mice. However, a slight trend toward reduced Chat transcripts in the sampled tissues in the Tx group suggested an additional effect of loxP recombination on the level of Chat transcripts.

Although the ICV injections at birth were the sole route of administration of the AAV-CHAT, qPCR analysis demonstrated a widespread distribution of the vector, especially in the CNS and liver, which is consistent with previous studies conducted in mice and nonhuman primates. By contrast the experiments using RT-qPCR showed that the expression of the transgene was highest in the CNS. This was likely the only source for ChAT translation because Chat transcripts in Tx-treated mice are incompetent to translate the intact Chat protein.

This preclinical study showed that the administration of AAV-CHAT to a Chat conditionally deficient mouse model through ICV injection at birth or IC-IP injections at the fourth week of life can ameliorate the motor deficit of this model and reestablish ChAT expression in SMNs safely without eliciting anomalous functions or inducing pathological changes in the organs of injected mice. The biodistribution of the vector in ICV-injected mice was widespread, but the expression of the transgene was highest in the CNS.

### Materials and Methods

### Development of a Conditional Chat^{flox/Flox} Model

Chat knockout (KO) mouse model: The ChAT knockout mouse model was developed in the laboratory of Dr. Joshua R. Sanes to study the role of neurotransmitters in synapse formation at the neuromuscular junction (NMJ) [9] and is commercially available at Jackson laboratories (B6; 129-Chat^{tm1Jrs}/(J) [5]. This model was developed by inserting a loxP site 700 bp upstream of exon 4 and another loxP site 200 bp downstream of exon 5, which is adjacent to an FRT site flanked PGK-Neo selection cassette (neo′) as shown in FIG. 2. Thus, these mice contain the FRT site flanked PGK-Neo selection cassette. It has been reported that the presence of the FRT site flanked PGK-Neo cassette has no detectable effect on gene expression. The breeding of these mice to a β-actin-Cre transgenic line generated Chat KO mice. However, Chat knockout mice are immobile and die at birth, thus not allowing postnatal therapeutic interventions. In contrast with the β-actin-Cre crossing, the breeding of Chat^{flox/flox} mice to Cre-ER^{T2} generates mice that develop Chat deficiency only when they are exposed to tamoxifen. Thus, the model provides the opportunity for therapeutic interventions using AAV-mediated replenishment of the enzyme. FIG. 2 depicts a diagram showing exons 4 and 5 contained between two LoxP sites and a downstream FRT flanked Neo selection cassette. The positions of primers for mouse genotyping are shown in blue [a-b: mutant (MT); a-c: wild type (WT)]. ATG represents the translational start. FIG. 2 was modified from Misgeld et al. (2002) Neuron., 36:635-48 [5].

Conditional Cre-ER^{T2} (B6.129-Gt(ROSA)26Sor^{tm1(cre/ERT2)Ty}): A conditional Cre-ERT2 (Cre recombinase-estrogen receptor T2) was developed by introducing the selection cassette into the estrogen receptor T2 gene [10-12]. In this model the ERT2 retains the Cre recombinase in the cytoplasm until tamoxifen administration releases inhibition, thus permitting the recombination of genomic loxP sites in the nucleus. Tamoxifen-induced Cre-mediated recombination throughout the body has been demonstrated through crossing with a Cre-responsive beta galactosidase reporter strain. This conditional model is available at Jackson Laboratories. After several breeding stages of Chat^{flox/flox} to Cre-ER 72 mice with the ChAT^{flox/flox}/Cre-ER^{T2/T2} genotype were obtained on a C57BL/6 background, which constitutes the genetic composition of the mice colony. Double transgenic mice for Chat^{flox/flox} and Cre-ER^{T2} are normal at birth, develop normally and are fertile. However, if they receive two intraperitoneal (IP) injections of tamoxifen 40 mg/Kg 6-8 h apart at postnatal day 11 (P11) their growth arrests, they became progressively weak, hunched back, and they all die.

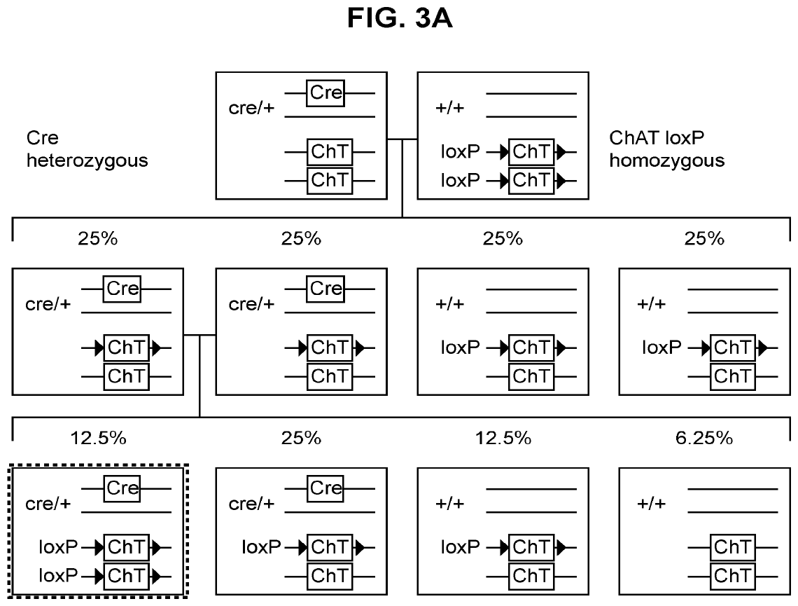

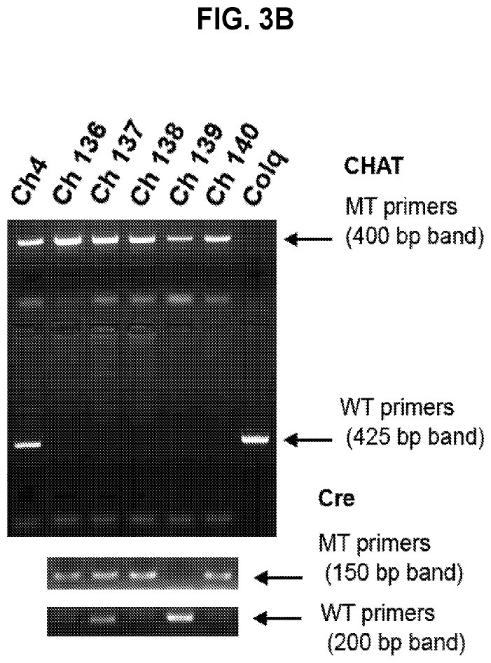

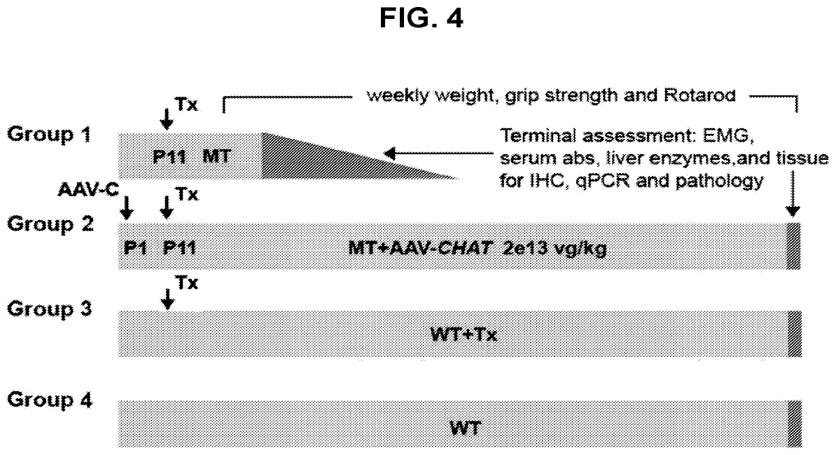

Breeding strategy: Several breeding stages were completed starting from a homozygous ChAT^{flox/flox} mouse bred to a heterozygous Cre-ER^{T2} (FIG. 3A). The first stage provided 50% of mice with the ChAT^{flox/WT}/Cre-ER^{T2} genotype. The inbreeding of these animals resulted in 12.5% mice with the target genotype ChAT^{flox/flox}/Cre-ER^{T2/WT} (red square). A female and a male with the ChAT^{flox/flox}/Cre-ER^{T2/WT} genotype were separated and used as breeders. The offspring of these mice were expected to produce 50% ChAT^{flox/flox}/Cre-ER^{T2/WT} and 25% ChAT^{flox/flox}/Cre-ER^{T2/T2}. FIG. 3 shows that mice 136, 137, 138 and 140 carry the desired ChAT^{flox/flox}/Cre-ER^{T2/WT} or ChAT^{flox/flox}/Cre-ER^{T2/T2} genotype. Recombination of LoxP sites using tamoxifen injection can be induced in these mice. FIG. 3A depicts the breeding strategy. The desired genotype is within the red square. FIG. 3B shows genotyping using DNA from the tail of the mice. Mouse Ch4 is a ChAT^{flox/WT} (amplified by MT and WT primers). Mice Ch 136-140 are ChAT^{flox/flox} (only amplified by MT primers) (top). Colq is ChAT^{WT/WT} (only amplified by WT primers). Mice Ch 136-138 and Ch 140 are either ChAT^{flox/flox}/Cre-ER^{T2/WT} (Ch 137) or ChAT^{flox/flox}/Cre-ER^{T2/T2} (Ch 136, 138 and 140) (amplified only by MT Cre primers).

### Development of the AAV-CHAT Vector

The development of the AAV vector was performed through Genecopoeia Laboratories by cloning the human ChAT gene (CHAT), CHAT-T2A-GFP in a rAAV cis vector (the pEZ-AV06 vector) under the CBh promoter, resulting in the CS-T2064-AV06-01 vector. The AAV9 is selected as a carrier because of the well-known biodistribution of this serotype in the CNS, including spinal cord motor neurons. The human CHAT rather than the mouse Chat was selected to facilitate the transition to future studies in humans. The open reading frame (ORF) for human CHAT is 1,893 bp. The CBh promoter was chosen because it provides strong, long-term, and ubiquitous CNS expression and because it does not silence with time as other promoters like CMV. The size of the CBh promoter is 800 bp [13]. CHAT was cloned single stranded (ss) to allow space for EGFP at the cloning site (total 4.7 kb). EGFP was included in the first construct to facilitate the detection of the expression of the vector in the CNS. Next generation constructs will not include EGFP in preparation for human studies, to potentially allow package of an inverted self-complementary (sc) repeat of CHAT to increase the potency of the vector [12-15], and also because the expression of the construct in animal models is followed up with quantitative PCR (qPCR). The size of the EGFP gene is 730 bp.

Briefly the manufacturing process involves plasmid development, cell expansion, plasmid transfection, and viral vector collection. The cloning step was followed by AAV packaging (including the rep/cap-gene containing and pHelper plasmids), purification, and viral titration of >1E+13 VG/mL (100 uL). For the development of the AAV expression plasmid, the construction is verified using both gene-specific and vector specific primers as well as enzyme digestion. The AAV expression construct is validated by full-length sequencing, restriction enzyme digestion and PCR-size validation using gene-specific and vector-specific primers. The AAV packaging plasmids are validated by enzyme digestions. The AAV particles are confirmed free of bacteria, fungi, and common Mycoplasma contamination. The purification of the AAV9 particles is done using the partition method. The titration of the AAV particles is performed using real time quantitative PCR. Because of the well-known decline of AAV titers with time, transportation, freezing and thawing, all experiments involving AAV9-CHAT are preceded by a repeat viral titration. This is done using real-time PCR and primers targeting the HGH polyadenylation signal.

### AAV-CHAT Vector Dose

To determine the optimal dose of the single-stranded (ss) AAV9-CBh-CHAT vector that can improve weakness and protect against lethality in mice conditionally deficient of the enzyme choline acetyltransferase, three doses for intracranial (IC) injection were tested at P1: 8e12 vg/kg, 2e13 vg/kg and 3.3e13vg/kg.

The dose of 8e12 vg/kg could not prevent lethality in mutant mice later injected with Tx. By contrast, all pups injected with the dose of 3.3e13 vg/kg died within 48 h after injection. Thus, the dose of 2e13vg/kg was deemed to be the most appropriate dose and all AAV injected mice at P1 received that dose.

A lower dose for the AAV vector may be possible by using a self-complementary AAV (scAAV) generated by cloning an inverted sequence of CHAT to allow self-complementary transcription. In the current vector, the size of the CHAT (1,893 bp) does not allow the inclusion of the GFP gene and two copies of CHAT at the same time. However, since in the gene therapy vector, GFP will be excluded, an inverted sequence of CHAT can be added to allow generation of scAAV.

### Behavioral Studies

Electromyogram: Needle electromyogram (EMG) recordings from the soleus-gastrocnemius muscles of either side during electrical stimulation of the sciatic nerve at 2, 10, 30 and 50 Hz were performed before and after a long train of stimuli at 30 Hz for 30 seconds.

Immunohistochemistry: The spinal cords from mice were extracted, fixed in 2.5% paraformaldehyde, exposed for 3-5 days to 30% sucrose and sliced in a cryostat at the cervical and lumbar levels. The slices were exposed for 48 h to a primary antibody against ChAT using the free-floating technique. Slides with mounted spinal cord section were viewed with a Zeiss invertoscope and ChAT immunofluorescence was quantified using the ImageJ program.

Grip strength: The grip strength of fore and hind limbs was performed using the Chatillon DFE-II force transducer (Ametek, Beijing, China). Mice were placed on a metal grid connected to the force transducer and gently pulled from the tail while the force was digitally recorded.

Rotarod: The rotarod test was performed using the Rotamex instrument (Columbus Instruments, Columbus, OH, USA). Mice underwent three training sessions a week before the start of the data collection. After the week of training mice performed three trials per session. During each trial, mice were placed on a rotating rod that accelerated every 6 s by 1 rpm from 0 to 40 rpm. The rpm and time of fall were recorded at each trial.

Viral genome biodistribution: DNA was extracted from tissues using QIAamp columns (Cat. No. 51306; Qiagen). The copy numbers of the viral genome (vg) were calculated using the absolute quantification method based on a standard curve generated by quantitative (q) PCR of a synthesized amplicon with a known number of molecules per weight. The biodistribution of the AAV9 in peripheral organs measured by qPCR is particularly relevant in the DRG, since involvement of the DRG could result in sensory ataxia; and in the liver, because of the well-known liver toxicity of AAV-treatment [14]. The effect of IC administration of the AAV9-hCHAT on liver toxicity is being assessed.

Necropsy and histology: Mice were deeply anesthetized with isoflurane and euthanized by opening the chest cavity and inducing a pneumothorax while the heart was perfused with saline solution followed by 4% paraformaldehyde (PFA). Tissues were harvested from the heart, liver, colon, brain, spinal cord, and dorsal root ganglia and immersed in PFA. Histological analysis of key structures of the CNS, as well as key peripheral organs, was performed to assess for inflammation such cellular infiltrates and fibrinoid necrosis reported in other species injected with AAV [9].

Statistical methods: Graphed data show median values plus minus standard error of the means (Prism; GraphPad, San Diego, CA, USA). Statistical significance was evaluated using the two-way analysis of variance (ANOVA) or the two-tail Student's t-test. Weight and EMG decrements were evaluated using the chi-squared test. For analysis of the survivability between treatment groups, significance was evaluated using the Kaplan-Meier curve. If a significant effect is demonstrated by the overall ANOVA tests, the multivariant Bonferroni post-hoc test will be utilized to determine specific group differences.

Immunologic response of the host: These experiments will consist of measuring neutralizing antibodies in the serum of mice at the time of the terminal studies. Briefly, 293T cells are maintained in DMEM with 10% FBS and 1% penicillin/streptomycin at 37° C. and 5% CO₂. 2× 10⁴ cells/well in DMEM+penicillin/streptomycin are seeded into 96 well-plates the day of the infection. On the same day two-fold serial dilutions of the animal sera in DMEM are prepared in a separate 96 well-plate, Wells with DMEM only are used as controls. Next, 2×108/well vg of AAV-Luc are added to the sera samples and DMEM control wells, incubated at 37° C. for 30 minutes and then added to the 293T cells. The transduction efficiency is analyzed 24 h later by quantifying luciferase activity. The percentage of inhibition is calculated relative to the DMEM control. Additionally, the presence of antibodies against the AAV9 capsid in the serum of mice that have received the AAV9-hCHAT treatment will be tested.

Correlating clinical response with ChAT expression: The clinical response will be correlated with the expression of ChAT in the spinal cord using quantitative immune-histochemistry and immunoblotting. The clinical response and ChAT expression will be correlated with the AAV9 genome copy number measured by qPCR in tissues from several areas of the CNS, including the spinal cord, frontal cortex and cerebellum. To accomplish this, the methodology for ChAT immunohistochemistry in the spinal cord briefly described above will be used. In addition, quantitative Western blotting will be used. The biodistribution of the vector will be measured in the CNS, DRG, heart and liver using qPCR.

### Example 3: Human Clinical Trials

Congenital myasthenia due to CHAT mutations is one of the multiple variants of congenital myasthenic syndromes (CMS), which are rare diseases characterized by weakness and muscle fatigue. After performing the above experiments, it is believed that the administration of the described gene therapy product will either cure the disease or change the course of the illness to the extent that it will prevent threatening episodes of apneas in the mild forms and will reduce the on-time on the respirator in the severe forms of the disease. The described AAV gene therapy product will reprogram cholinergic neurons of the central nervous system (CNS), including the spinal motor neuron (MN) that innervates the neuromuscular junction, to produce a wild type (WT) catalytic efficient ChAT.

In the group of patients with neonatal onset CMS for whom the initial AAV trial is intended, the disease is very homogeneous. In three observed patients with neonatal onset: all required tracheostomy, a gastric tube, and mechanical ventilation 24/7. Additionally, none of the patients were able to ambulate independently, all were wheelchair-bound, and all showed severe delay of motor developmental milestones. Further, two out of three patients died of complications of the disease.

CMS patients can be diagnosed by clinical assessment by a neonatologists or pediatrician and consultation with a neurologist/neuromuscular specialist; and optionally electrodiagnostic with EMG and repetitive nerve stimulation. Additionally, genetic studies covering a large number of genes with proven association with CMS using Next Generation Sequencing (NGS) may be employed to confirm a CMS diagnosis.

Additional endpoints and outcomes for determining the effectiveness of the described gene therapy product in treating CMS may include reductions of apneic episodes, increased time-off mechanical ventilation, change from baseline to end of study in the Myasthenia Gravis Activities of Daily Living (MG-ADL) total score, and change from baseline to end of study in the Quantitative Myasthenia Gravis (QMG) total score. Further, given the severe motor and respiratory involvement of these patients many of the endpoints used for patients with SMA could be used for patients with CHAT-CMS, including ability to sit independently, ability to overcome gravity with proximal muscles, ability to stand up and ambulate and independency or time-off mechanical ventilation. Because the mortality is very high, survival can also be used as a co-primary endpoint. Secondary endpoints may include subjective assessment of quality of life.

### Example 4: Induced Pluripotent Stem Cells (iPSCs) for Correcting CHAT Mutations

As demonstrated in the previous examples, congenital myasthenia due to CHAT mutations may be cured or otherwise treated by delivering the described AAV gene therapy product in order to reprogram cholinergic neurons of the CNS, including the spinal MN that innervates the neuromuscular junction, to produce a WT catalytic efficient ChAT. Alternatively, induced pluripotent stem cells (iPSCs) may be used to produce a WT catalytic efficient ChAT in a patient in order to cure or otherwise treat congenital myasthenia resulting from CHAT mutations. The use of iPSCs for congenital myasthenia treatment may be tested on mouse models (e.g., as described above) before beginning human clinical trials.

iPSCs may be generated from a patient's own cells (such as, e.g., a patient's own skin cells), which will be reprogrammed to iPSCs. These are then differentiated into patient-specific, cholinergic neurons (or, e.g., acetylcholine-producing neuronal precursor cells) before transplantation into the patient's brain. The iPSCs will be genetically edited or otherwise produced in a manner to ensure CHAT mutations are not present in the cells transplanted in the patient's brain. For example, CRISPR-editing can be used to ensure patient derived iPSCs are free of disease-causing CHAT mutations. The donor cells may be injected directly into the brain as cell suspensions or cell clumps through a transplantation needle.

### REFERENCES

[1] Abicht A, Müller J S, Lochmüller H. Congenital Myasthenic syndromes. 2003 May 9 [updated 2016 Jul. 14]. In: Adam M P, Ardinger H H, Pagon R A, Wallace S E, Bean L J H, Mefford H C, Stephens K, Amemiya A, Ledbetter N, editors. GencReviews® [internet]. Seattle (WA): University of Washington, Seattle; 1993-2017. Available from http://www.ncbi.nlm.nih.gov/books/NBK1168/.
[2] Engel, A. G., Shen, X. M., Selcen, D., & Sine, S. M. (2015). Congenital myasthenic syndromes: Pathogenesis, diagnosis, and treatment. Lancet Neurology, 4, 420-434. PMID: 25792100.
[3] Finsterer J. Congenital myasthenic syndromes. Orphanet J Rare Dis. 2019 Feb. 26; 14 (1): 57. doi: 10.1186/s13023-019-1025-5.
[4] Schara U, Christen H J, Durmus H, Hietala M, Krabetz K, Rodolico C, Schreiber G, Topaloglu H, Talim B, Voss W, Pihko H, Abicht A, Müller J S, Lochmüller H. Long-term follow-up in patients with congenital myasthenic syndrome due to CHAT mutations. Eur J Paediatr Neurol. 2010 July; 14 (4): 326-33.
[5] Maselli R A, Chen D, Mo D, Bowe C, Fenton G, Wollmann R L. Choline acetyltransferase mutations in myasthenic syndrome due to deficient acetylcholine resynthesis. Muscle Nerve. 2003 February; 27 (2): 180-7.
[6] Conomy J P, Levinsohn M, Fanaroff A. Familial infantile myasthenia gravis: a cause of sudden death in young children. J Pediatr. 1975 September; 87 (3): 428-30.
[7] Arredondo J, Lara M, Gospe S M Jr, Mazia C G, Vaccarezza M, Garcia-Erro M, Bowe C M, Chang C H, Mezei M M, Maselli R A. Choline Acetyltransferase Mutations Causing Congenital Myasthenic Syndrome: Molecular Findings and Genotype-Phenotype Correlations. Hum Mutat. 2015 September; 36 (9): 881-93.
[8] Shen X M, Crawford T O, Brengman J, Acsadi G, Iannaconne S, Karaca E, Khoury C, Mah J K, Edvardson S, Bajzer Z, Rodgers D, Engel A G. Functional consequences and structural interpretation of mutations of human choline acetyltransferase. Hum Mutat. 2011 November; 32 (11): 1259-67.
[9] Misgeld T, Burgess R W, Lewis R M, Cunningham J M, Lichtman J W, Sanes J R. Roles of neurotransmitter in synapse formation: development of neuromuscular junctions lacking choline acetyltransferase. Neuron. 2002 Nov. 14; 36 (4): 635-48.
[10] Ventura A, Kirsch D G, Mclaughlin M E, Tuveson D A, Grimm J, Lintault L, et al. Restoration of p53 function leads to tumour regression in vivo. Nature. 2007; 445:661-5.
[11] Valny M, Honsa P, Kirdajova D, Kamenik Z, Anderova M. Tamoxifen in the Mouse Brain: Implications for Fate-Mapping Studies Using the Tamoxifen-Inducible Cre-loxP System. Front Cell Neurosci. 2016 Oct. 20; 10:243.
[12] Imai T, Jiang M, Chambon P, Metzger D. Impaired adipogenesis and lipolysis in the mouse upon selective ablation of the retinoid X receptor alpha mediated by a tamoxifen-inducible chimeric Cre recombinase (Cre-ERT2) in adipocytes. Proc Natl Acad Sci USA. 2001 Jan. 2; 98 (1): 224-8.
[13] Gray S J, Foti S B, Schwartz J W, Bachaboina L, Taylor-Blake B, Coleman J, Ehlers M D, Zylka M J, McCown T J, Samulski R J. Optimizing promoters for recombinant adeno-associated virus-mediated gene expression in the peripheral and central nervous system using self-complementary vectors. Hum Gene Ther. 2011 September; 22 (9): 1143-53.
[14] Hinderer C, Katz N, Buza E L, Dyer C, Goode T, Bell P, Richman L K, Wilson J M.

Severe Toxicity in Nonhuman Primates and Piglets Following High-Dose Intravenous Administration of an Adeno-Associated Virus Vector Expressing Human SMN. Hum Gene Ther. 2018 March; 29 (3): 285-298.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

What is claimed is:

1. A method for treating a neuromuscular junction (NMJ) disorder in a subject, the method comprising administering to the subject a therapeutically effective dose of a recombinant adeno-associated virus (rAAV) virion comprising a nucleic acid encoding choline acetyltransferase (ChAT), wherein administering the rAAV virion results in expression of the ChAT in the subject thereby treating the NMJ disorder in the subject.

2. The method of claim 1, wherein the NJM disorder is a congenital myasthenic syndrome (CMS) or myasthenia gravis (MG).

3. The method of claim 2, wherein the NJM disorder is a CMS.

4. The method of claim 3, wherein the CMS is a presynaptic CMS.

5. The method of claim 3 or 4, wherein the CMS is CMS with episodic apnea (CMS-EA).

6. The method of any one of claims 2 to 5, wherein the CMS is associated with a mutation in the endogenous CHAT gene of the subject.

7. The method of claim 6, wherein the mutation reduces the enzymatic activity of ChAT in the subject.

8. The method of claim 7, wherein the mutation is Val136Met, Arg186Trp, Val194Leu, Arg207His, Pro211Ala, Arg566Cys, and/or Ser694Cys, wherein the amino acid numbering is based on the amino acid sequence set forth in SEQ ID NO:1.

9. The method of claim 8, wherein the mutation is Val136Met and/or Arg207His.

10. The method of one of claims 1-9, wherein the subject is eight years of age or younger and has a body weight sufficient to allow the administration of an effective dose of AAV safely.

11. The method of claim 10, wherein the subject is an in-utero fetus or a newborn.

12. The method according to any one of claims 1-11, wherein the subject is a human and the ChAT is a human ChAT.

13. The method of any one of claims 1-12, wherein the rAAV virion comprises AAV-retro, AAV-Rh10, AAV-PHP.B, AAV-PHP.eB, AAV-PHP.S or AAV-Anc80 capsid proteins.

14. The method of any one of claims 1 to 12, wherein the rAAV virion comprises AAV9 capsid proteins.

15. The method of any one of claims 1-14, wherein the rAAV virion comprises a nucleic acid comprises an AAV inverted terminal repeat (ITR), a promoter operably linked to the nucleic acid encoding ChAT, and an AAV ITR.

16. The method of claim 15, wherein the promoter exhibits strong, ubiquitous, long-term expression in the central nervous system (CNS) of the subject.

17. The method of claim 16, wherein the promoter is a CBA hybrid intron (CBh) promoter.

18. The method of any one of claims 15-17, wherein the nucleic acid encoding ChAT encodes a single stranded ChAT.

19. The method of any one of claims 15-17, wherein the nucleic acid encoding ChAT comprises an inverted self-complementary sequence of the ChAT such that a double stranded sequence encoding the ChAT is produced in a cell of the subject following the administration.

20. The method of any one of claims 15-19, wherein nucleic acid comprises an rAAV vector, and the vector is AAV-2, AAV-retro, AAV-Rh10, AAV-PHP.B, AAV-PHP.eB, AAV-PHP.S, AAV-Anc80, AAV-9, or a vector derived therefrom.

21. The method of any one of claims 15-20, wherein the ITRs are AAV2 ITRs, AAV3 ITRs, or AAV9 ITRs, optionally, wherein the AAV ITRs are AAV2 ITRs.

22. The method according to any one of claims 15-19, wherein the rAAV virion comprises a nucleic acid comprising at least 70%, 75%, 80%, 85%, 90%, 95%, or 100% sequence identity to SEQ ID NO:11 or SEQ ID NO:12.

23. The method according to claim 22, wherein the rAAV virion comprises an AAV9 capsid.

24. The method of any one of claims 1-23, wherein the rAAV virion is administered by intravenous injection.

25. The method of any one of claims 1-24, wherein the rAAV virion is administered directly into the CNS.

26. The method of claim 25, wherein the administration into the CNS is via an intrathecal, intraventricular, or intracranial route.

27. The method of any one of claims 1-26, wherein the therapeutically effective dose of the rAAV virion comprises a dose of less than 2×10¹⁴ viral genomes per kilogram (vg/kg).

28. The method of claim 26, wherein the therapeutically effective dose of the rAAV virion comprises a dose of about 8×10¹², 2×10¹³, or 3.3×10¹³ viral genomes per kilogram (vg/kg).

29. The method of any one of claims 1-28, wherein the rAAV virion is administered with a pharmaceutically acceptable diluent, carrier or excipient.

30. The method of any one of claims 1-29, wherein the rAAV virion is administered to the subject on two or more occasions.

31. A recombinant adeno-associated viral (rAAV) virion comprising a nucleic acid encoding a choline acetyltransferase (ChAT).

32. The rAAV virion of claim 31, wherein the rAAV virion comprises a nucleic acid comprising an AAV inverted terminal repeat (ITR), a promoter operably linked to a nucleic acid sequence encoding choline acetyltransferase (ChAT), and an AAV ITR.

33. The rAAV virion of claim 31, wherein the ChAT is a human ChAT.

34. The rAAV virion of claim 32 or 33, wherein the rAAV virion comprises AAV2, AAV-retro, AAV-Rh10, AAV-PHP.B, AAV-PHP.eB, AAV-PHP.S or AAV-Anc80 capsid proteins.

35. The rAAV virion of claim 32 or 33, wherein the rAAV virion comprises AAV9 capsid proteins.

36. The rAAV virion of any one of claims 32-35, wherein the rAAV vector is AAV-2, AAV-retro, AAV-Rh10, AAV-PHP.B, AAV-PHP.eB, AAV-PHP.S, AAV-Anc80, AAV-9, or a vector derived therefrom.

37. The rAAV virion of any one of claims 32-35, wherein the ITRs are AAV2 ITRs, AAV3 ITRs, or AAV9 ITRs, optionally, wherein the AAV ITRs are AAV2 ITRs, the promoter is a CAG promoter, a CMV promoter, a CBA promoter, or a CBh promoter and the rAAV comprises AAV9 capsid.

38. The rAAV virion of any one of claims 32-35, wherein the rAAV virion comprises a nucleic acid comprising at least 70%, 75%, 80%, 85%, 90%, 95%, or 100% sequence identity to SEQ ID NO:11 or SEQ ID NO:12.

39. The rAAV virion of claim 38, wherein the rAAV virion comprises AAV9 capsid proteins.

40. The rAAV virion of any one of claims 32-39, wherein the promoter exhibits strong, ubiquitous, long-term expression in the central nervous system (CNS) of the subject.

41. The rAAV virion of claim 40, wherein the promoter is a CBA hybrid intron (CBh) promoter.

42. The rAAV virion of any one of claims 32 to 41, wherein the nucleic acid sequence encoding ChAT produces a single stranded sequence encoding the ChAT.

43. The rAAV virion of any one of claims 32 to 41, wherein the nucleic acid sequence encoding ChAT comprises an inverted self-complementary sequence of the ChAT such that a double stranded sequence encoding the ChAT is produced in a cell of a subject following administration of the rAAV virion.

44. A composition comprising the rAAV virion of any one of claims 31 to 43 and a pharmaceutically acceptable diluent, carrier, or excipient.

45. The composition of claim 44, wherein the composition comprises a therapeutically effective dose of the rAAV virion.

46. The composition of claim 45, wherein administration of the therapeutically effective dose results in an expression level of ChAT activity that is at least 50% of the normal ChAT activity level.

47. The composition of claim 45, wherein administration of the therapeutically effective dose results in a reduction of apneic episodes.

48. The composition of any one of claims 44-47, wherein the composition comprises about 2.4×10¹³, 6×10¹³, or 9.9×10¹³ viral genomes (vg).


SEQUENCE LISTING (12 sequences)

SEQ ID NO: 1 - protein, 630 aa, Homo sapiens
MAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHMTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 2 - DNA, 2244 bp, Homo sapiens
  source 1..2244: mol_type = genomic DNA
atggggctgaggacagcgaagaagagggggcttgggggaggggggaaatggaagagagaggagggaggaggtacaagaggaaggagagaagtgcggccagcttgctttctccagtcgggtggccgcggggacccgggcgacgtcggaggccctgccgggaacccaggctgcagcccccacccccgcgctgcgacacgccccccaccccttccggctcacacccccgcccacactcctgagtggtgcggtgcagcgtcggccgaggcagcagagccgaggagagcaggtccacacctctgcatccctgcaccaggactcaccaagacgcccatcctggaaaaggtcccccgtaagatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaccggagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacggcccaggaggcgtggagctcagcgacacccacagggcactccagctccttcacggcggaggctacagcaagaacggggccaatcgctggtacgacaagtccctgcagtttgtggtgggccgagacgcgacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccaccgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaacct

SEQ ID NO: 3 - DNA, 141 bp, Adeno-associated virus type 2
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 4 - DNA, 141 bp, Adeno-associated virus type 2
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 477 bp, Homo sapiens
  source 1..477: mol_type = genomic DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 6 - DNA, 222 bp, Simian vacuolating virus 40
  source 1..222: mol_type = genomic DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagtggcgtaatcat

SEQ ID NO: 7 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - DNA, 56 bp, Oryctolagus cuniculus
  source 1..56: mol_type = genomic DNA
aataaaggaaatttattttcattgcaatagtgtgttggaattttttgtgtctctca

SEQ ID NO: 9 - DNA, 793 bp, synthetic construct
  source 1..793: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 10 - DNA, 464 bp, Homo sapiens
  source 1..464: mol_type = genomic DNA
gagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 11 - DNA, 2974 bp, synthetic construct
  source 1..2974: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggccaccatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccatcgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 2645 bp, synthetic construct
  source 1..2645: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccatcgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg